(19) United States
(12) Patent Application Publication
(10) Pub. No.: US 2002/0055474 A1
(43) Pub. Date: May 9, 2002

(54) NOVEL MOLECULES OF THE TNF LIGAND SUPERFAMILY AND USES THEREFOR

(76) Inventor: SAMANTHA J. BUSFIELD, CAMBRIDGE, MA (US)

Correspondence Address: DEANN F. SMITH, LAHIVE AND COCKFIELD, LLP, 28 STATE STREET, BOSTON, MA, 02109, US

(21) Appl. No.: 09/014,348

(22) Filed: Jan. 27, 1998

(51) Int. Cl. A61K 48/00; C07H 21/04; C12N 15/00; C12P 21/04

(52) U.S. Cl. 514/44; 435/70.1; 435/320.1; 435/325; 435/455; 536/23.5

(57) ABSTRACT

Novel TRASH polypeptides, proteins, and nucleic acid molecules are disclosed. In addition to isolated, full-length TRASH proteins, the invention further provides isolated TRASH fusion proteins, antigenic peptides and anti-TRASH antibodies. The invention also provides TRASH nucleic acid molecules, recombinant expression vectors containing a nucleic acid molecule of the invention, host cells into which the expression vectors have been introduced and non-human transgenic animals in which a TRASH gene has been introduced or disrupted. Diagnostic, screening and therapeutic methods utilizing compositions of the invention are also provided.

### BACKGROUND OF THE INVENTION

Cytokines are small peptide molecules produced by a variety of cells that mediate a wide range of biological activities. Arai, K.-I. et al. (1990) Annu. Rev. Biochem. 59:783 and Paul, W. E. and R. A. Seder (1994) Cell 76:241. Through a complex network, cytokines regulate functions including cellular growth, inflammation, immunity, differentiation and repair. Mosmann, T. R. (1991) Curr. Opin. Immunol. 3:311. One family of cytokines, includes the tumor necrosis factor ligand (TNFL) superfamily of proteins which includes two structurally and functionally related proteins, TNF-α and TNF-β. TNF-α (also known as cachectin) is synthesized as a type II membrane protein which then undergoes post-translational cleavage liberating the extracellular domain. TNF-α possesses a wide variety of functions including the ability to induce cytolysis of certain tumor cell lines and the induction of cahexia. TNF-α is a potent pyrogen, causing fever by direct action or by stimulation of interleukin-1 secretion. In addition, TNF-α can stimulate cell proliferation and induce cell differentiation under certain conditions. TNF-α is characteristically produced at the sites of inflammation by infiltrating mononuclear cells. TNF-α plays a beneficial role as an immunostimulant and an important mediator of host resistance to many infectious agents. Overproduction of TNF-α can lead to severe systemic toxicity and even death. TNF-α has also been implicated in the pathogenesis of some autoimmune disorders.

CD27L, CD30L, CD40L, FASL, Lt-β, and 4-1BBL also appear to be type II membrane proteins. All of these cytokines appear to form homotrimeric complexes that are recognized by their specific receptors.

Given members of the TNFL superfamily of proteins are involved in the activation of a large array of cellular genes and of multiple signal transduction pathways, kinases and transcription factors, there exists a need for the identification of novel TNFL-like molecules, as well as, for modulators of such molecules for use in regulating a variety of cellular responses.

### SUMMARY OF THE INVENTION

The present invention is based, at least in part, on the discovery of novel members of the TNF ligand superfamily, referred to herein as TRASH nucleic acid and protein molecules. The TRASH molecules of the present invention are useful as modulating agents in regulating a variety of cellular processes. Accordingly, in one aspect, this invention provides isolated nucleic acid molecules encoding TRASH proteins or biologically active portions thereof, as well as nucleic acid fragments suitable as primers or hybridization probes for the detection of TRASH encoding nucleic acids.

In one embodiment, a TRASH nucleic acid molecule is 60% homologous to the nucleotide sequence shown in SEQ ID NO:1, or a complement thereof In another embodiment, a TRASH nucleic acid molecule is 60% homologous to the nucleotide sequence shown in SEQ ID NO:3, SEQ ID NO:6, SEQ ID NO:8, SEQ ID NO:10, or a complement thereof In a preferred embodiment, an isolated TRASH nucleic acid molecule encodes the amino acid sequence of human TRASH.

In a preferred embodiment, an isolated TRASH nucleic acid molecule has the nucleotide sequence of SEQ ID NO:3 or a complement thereof. In another embodiment, a TRASH nucleic acid molecule further comprises nucleotides 1-272 of SEQ ID NO:1. In yet another preferred embodiment, a TRASH nucleic acid molecule further comprises nucleotides 1026-1344 of SEQ ID NO:1.

In yet another preferred embodiment, an isolated TRASH nucleic acid molecule has the nucleotide sequence shown in SEQ ID NO:1, or a complement thereof. In still another preferred embodiment, an isolated TRASH nucleic acid molecule has the nucleotide sequence shown in SEQ ID NO:3, SEQ ID NO:6, SEQ ID NO:8, or SEQ ID NO:10.

In another embodiment, a TRASH nucleic acid molecule includes a nucleotide sequence encoding a protein having an amino acid sequence sufficiently homologous to the amino acid sequence of SEQ ID NO:2, SEQ ID NO:7, SEQ ID NO:9, or SEQ ID NO:11. In yet another embodiment, a TRASH nucleic acid molecule includes a nucleotide sequence encoding a protein having an amino acid sequence at least 60% homologous to the amino acid sequence of SEQ ID NO:2, SEQ ID NO:7, SEQ ID NO:9, or SEQ ID NO:11. In a preferred embodiment, a TRASH nucleic acid molecule includes a nucleotide sequence encoding a protein having the amino acid sequence of SEQ ID NO:2, SEQ ID NO:7, SEQ ID NO:9, or SEQ ID NO:11.

In another embodiment, an isolated nucleic acid molecule of the present invention encodes a TRASH protein which includes a TNF signature motif. In another embodiment, an isolated nucleic acid molecule of the present invention encodes a TRASH protein which includes a TNF signature motif and aTNF-like N-terminal transmembrane anchor for a type II membrane protein. In another embodiment, the TRASH nucleic acid molecule encodes a TRASH protein and is a naturally occurring nucleotide sequence. In yet another embodiment, an isolated nucleic acid molecule of the present invention encodes a TRASH protein and comprises a nucleotide sequence which hybridizes under stringent hybridization conditions to a nucleic acid molecule having the nucleotide sequence of SEQ ID NO:1, SEQ ID NO:3, SEQ ID NO:6, SEQ ID NO:8, or SEQ ID NO:10.

Another embodiment of the invention features TRASH nucleic acid molecules which specifically detect TRASH nucleic acid molecules relative to nucleic acid molecules encoding non-TRASH proteins. For example, in one embodiment, a TRASH nucleic acid molecule hybridizes under stringent conditions to a nucleic acid molecule comprising nucleotides 273-1025 of SEQ ID NO:1. In another embodiment, the TRASH nucleic acid molecule is at least 500 nucleotides in length and hybridizes under stringent conditions to a nucleic acid molecule comprising the nucleotide sequence shown in SEQ ID NO:1, SEQ ID NO:3, SEQ ID NO:6, SEQ ID NO:8, SEQ ID NO:10, or a complement thereof.

Another embodiment the invention provides an isolated nucleic acid molecule which is antisense to the coding strand of a TRASH nucleic acid.

Another aspect of the invention provides a vector comprising a TRASH nucleic acid molecule. In certain embodiments, the vector is a recombinant expression vector. In another embodiment the invention provides a host cell containing a vector of the invention. The invention also provides a method for producing TRASH protein by culturing in a suitable medium, a host cell of the invention containing a recombinant expression vector such that TRASH protein is produced.

Another aspect of this invention features isolated or recombinant TRASH proteins and polypeptides. In one embodiment, an isolated TRASH protein has a TNF signature motif. In another embodiment, an isolated TRASH protein has a TNF signature motif and a TNF-like N-terminal signal transmembrane anchor for a type II membrane protein. In a preferred embodiment, an isolated TRASH protein further comprises two cysteine residues that may be disulphide linked. In yet another preferred embodiment, an isolated TRASH protein further comprises two putative N-linked glycosylation sites.

In another embodiment, an isolated TRASH protein has an amino acid sequence sufficiently homologous to the amino acid sequence of SEQ ID NO:2, SEQ ID NO:7, SEQ ID NO:9, or SEQ ID NO:11. In a preferred embodiment, a TRASH protein has an amino acid sequence at least about 60% homologous to the amino acid sequence of SEQ ID NO:2, SEQ ID NO:7, SEQ ID NO:9, or SEQ ID NO:11. In another embodiment, a TRASH protein has the amino acid sequence of SEQ ID NO:2, SEQ ID NO:7, SEQ ID NO:9, or SEQ ID NO:11.

Another embodiment of the invention features an isolated TRASH protein which is encoded by a nucleic acid molecule having a nucleotide sequence at least about 60% homologous to a nucleotide sequence of SEQ ID NO:1, SEQ ID NO:3, SEQ ID NO:6, SEQ ID NO:8, SEQ ID NO:10, or a complement thereof. This invention also features an isolated TRASH protein which is encoded by a nucleic acid molecule having a nucleotide sequence which hybridizes under stringent hybridization conditions to a nucleic acid molecule comprising the nucleotide sequence of SEQ ID NO:1, SEQ ID NO:3, SEQ ID NO:6, SEQ ID NO:8, SEQ ID NO:10, or a complement thereof.

The TRASH proteins of the present invention, or biologically active portions thereof, can be operatively linked to a non-TRASH polypeptide to form TRASH fusion proteins. The invention further features antibodies that specifically bind TRASH proteins, such as monoclonal or polyclonal antibodies. In addition, the TRASH proteins or biologically active portions thereof can be incorporated into pharmaceutical compositions, which optionally include pharmaceutically acceptable carriers.

In another aspect, the present invention provides a method for detecting TRASH expression in a biological sample by contacting the biological sample with an agent capable of detecting a TRASH nucleic acid molecule, protein or polypeptide such that the presence of TRASH nucleic acid molecule, protein or polypeptide is detected in the biological sample.

In another aspect, the present invention provides a method for detecting the presence of TRASH activity in a biological sample by contacting the biological sample with an agent capable of detecting an indicator of TRASH activity such that the presence of TRASH activity is detected in the biological sample.

In another aspect, the invention provides a method for modulating TRASH activity comprising contacting the cell with an agent that modulates TRASH activity such that TRASH activity in the cell is modulated. In one embodiment, the agent inhibits TRASH activity. In another embodiment, the agent stimulates TRASH activity. In one embodiment, the agent is an antibody that specifically binds to TRASH protein. In another embodiment, the agent modulates expression of TRASH by modulating transcription of a TRASH gene or translation of a TRASH mRNA. In yet another embodiment, the agent is a nucleic acid molecule having a nucleotide sequence that is antisense to the coding strand of the TRASH mRNA or the TRASH gene.

In one embodiment, the methods of the present invention are used to treat a subject having a disorder characterized by aberrant TRASH protein or nucleic acid expression or activity by administering an agent which is a TRASH modulator to the subject. In one embodiment, the TRASH modulator is a TRASH protein. In another embodiment the TRASH modulator is a TRASH nucleic acid molecule. In yet another embodiment, the TRASH modulator is a peptide, peptidomimetic, or other small molecule. In a preferred embodiment, the disorder characterized by aberrant TRASH protein or nucleic acid expression is an immune disorder, a differentiative disorder, or a developmental disorder.

The present invention also provides a diagnostic assay for identifying the presence or absence of a genetic alteration characterized by at least one of (i) aberrant modification or mutation of a gene encoding a TRASH protein; (ii) mis-regulation of said gene; and (iii) aberrant post-translational modification of a TRASH protein, wherein a wild-type form of said gene encodes an protein with a TRASH activity.

In another aspect the invention provides a method for identifying a compound that binds to or modulates the activity of a TRASH protein, by providing a indicator composition comprising a TRASH protein having TRASH activity, contacting the indicator composition with a test compound, and determining the effect of the test compound on TRASH activity in the indicator composition to identify a compound that modulates the activity of a TRASH protein.

Other features and advantages of the invention will be apparent from the following detailed description and claims.

### BRIEF DESCRIPTION OF THE DRAWINGS

FIG. 1 depicts the cDNA sequence and predicted amino acid sequence of a human TRASH. The nucleotide sequence corresponds to nucleic acids 1 to 1344 of SEQ ID NO:1. The amino acid sequence corresponds to amino acids 1 to 250 of SEQ ID NO:2.

FIG. 2 depicts an alignment of the amino acid sequences of human TRASH I (corresponding to amino acids 1 to 250 of SEQ ID NO:2), human TNF-α (Swiss-Prot™ Accession No. PD01375), and human Tweak (Swiss-Prot™ Accession No. AF030099).

### DETAILED DESCRIPTION OF THE DRAWINGS

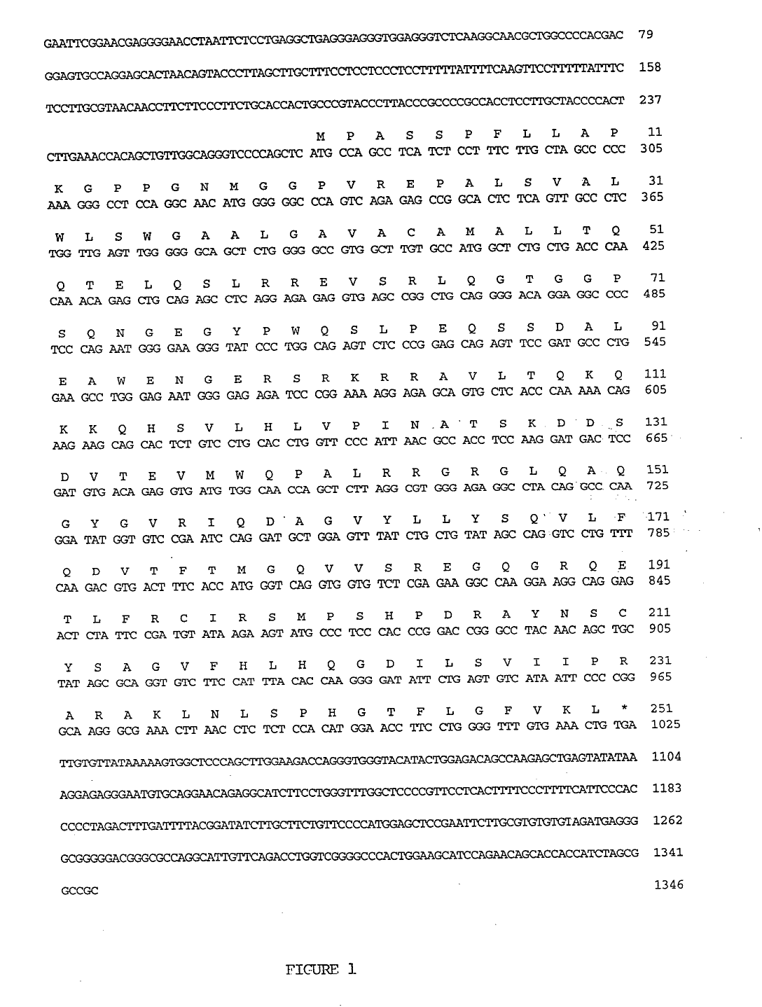

The present invention is based on the discovery of novel molecules having homology to members of the TNF ligand superfamily, referred to herein as TRASH protein and nucleic acid molecules, which comprise a family of molecules having certain conserved structural and functional features. The nucleotide sequence of a human TRASH nucleic acid molecule and the amino acid sequence of the respective human TRASH protein molecule are depicted in FIG. 1.

The term “family” when referring to the protein and nucleic acid molecules of the invention is intended to mean two or more protein or nucleic acid molecules having a common structural domain and having sufficient amino acid or nucleotide sequence homology as defined herein. Such family members can be naturally occurring and can be from either the same or different species. For example, a family can contain a first protein of human origin and a homologue of that protein of murine origin, as well as a second, distinct protein of human origin and a murine homologue of that protein. Members of a family may also have common functional characteristics.

In one embodiment, an TRASH family member is identified based on the presence of a “TNF signature motif” in the protein or corresponding nucleic acid molecule. As used herein, the term “TNF signature motif” refers to a protein domain which is a signature for the TNF family of proteins and contains the consensus sequence

V-Xaa₁-I-Xaa₂₍₁₋₃₎-GVYLL-Xaa₃₍₁₋₂₎-Q-V-Xaa₄-F

wherein V is the amino acid valine, Xaa₁ represents any amino acid, I is isoleucine, Xaa₂₍₁₋₃₎ represents 1 to 3 amino acids which can be the same or different, G is glycine, V is valine, Y is tyrosine, L is leucine, Xaa₃₍₁₋₂₎ represents 1 to 2 amino acids which can be the same or different, Q is glutamine, V is valine, Xaa₄ represents any amino acid, and F is phenylalanine (as represented in SEQ ID NO:12). In a preferred embodiment, the TRASH protein comprises a TNF signature motif which is at least about 10-20 amino acid residues in length, preferably at least about 12-18 amino acid residues in length, and more preferably at least about 15-17 amino acid residues in length. In another embodiment, the TRASH protein comprises a TNF signature motif which is at least about 55%, preferably at least about 65%, more preferably at least about 75-85% homologous, and even more preferably at least about 90-95% homologous to the TNF signature motif of SEQ ID NO:2 (about amino acids 155-171 or SEQ ID NO:4). In a preferred embodiment, the TRASH protein comprises the TNF signature motif of SEQ ID NO:2 (about amino acids 155-171 or SEQ ID NO:4).

In another embodiment, an TRASH family member is identified based on the presence of a TNF-like N-terminal signal transmembrane anchor for a type II membrane protein in the protein or corresponding nucleic acid molecule. As used herein, the term “TNF-like N-terminal signal transmembrane anchor for a type II membrane protein” refers to a protein domain located at the extreme N-terminal end of secretory and integral membrane proteins, contains a number of hydrophobic residues, and which serves to direct a protein containing such a sequence to a lipid bilayer. In a preferred embodiment, a TRASH protein comprises a TNF-like N-terminal signal transmembrane anchor for a type II membrane protein and contains at least 30-60, preferably about 35-55 amino acid residues, more preferably about 40-50 amino acid residues, still more preferably about 42-48 amino acid residues, and most preferably about 44-46 amino acid residues, of which at least about 40-70%, preferably about 50-65%, and more preferably about 55-60% are hydrophobic amino acid residues (e.g., Alanine, Valine, Leucine, Isoleucine, Phenylalanine, Tyrosine, Tryptophan, or Proline). In another preferred embodiment, a TRASH protein comprises a TNF-like N-terminal signal transmembrane anchor for a type II membrane protein which is at least about 55-60%, preferably at least about 65-70%, more preferably at least about 75-80%, still more preferably at least about 85-90%, and yet more preferably 5-98% homologous to the TNF-like N-terminal signal transmembrane anchor for a type II membrane protein shown in SEQ ID NO:2 (about amino acids 1-44 or SEQ ID NO:5). In still another preferred embodiment, a TRASH protein comprises a TNF-like N-terminal signal transmembrane anchor for a type II membrane protein of SEQ ID NO:2 (about amino acids 1-44 or SEQ ID NO:5).

In a preferred embodiment, a TRASH protein contains both a TNF signature motif and a TNF-like N-terminal signal transmembrane anchor for a type II membrane protein. In another preferred embodiment, a TRASH protein further contains two cysteine residues that may be disulphide linked. In another preferred embodiment, a TRASH protein further contains two putative N-linked glycosylation sites. In one exemplary embodiment, a TRASH protein contains a TNF signature motif including about amino acids 155-171 of SEQ ID NO:2. In another exemplary embodiment, a TRASH protein contains a TNF-like N-terminal signal transmembrane anchor for a type II membrane protein including about amino acids 1-44 of SEQ ID NO:2. In yet another exemplary embodiment, a TRASH protein further includes two cysteine residues that may be disulphide linked at amino acid 196 and 211 of SEQ ID NO:2. In still yet another exemplary embodiment, a TRASH protein further includes two putative N-linked glycosylation sites at about amino acid 124 and 237 of SEQ ID NO:2.

Preferred TRASH molecules of the present invention have an amino acid sequence sufficiently homologous to the amino acid sequence of SEQ ID NO:2, SEQ ID NO:7, SEQ ID NO:9, or SEQ ID NO:11. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains and/or a common functional activity. For example, amino acid or nucleotide sequences which share common structural domains have at least about 40% homology, preferably 50% homology, more preferably 60% -70% homology across the amino acid sequences of the domains and contain at least one, preferably two, and more preferably three or four structural domains, are defined herein as sufficiently homologous. Furthermore, amino acid or nucleotide sequences that share at least 40%, preferably 50%, more preferably 60, 70, or 80% homology and share a common functional activity are defined herein as sufficiently homologous.

As used interchangeably herein an “TRASH activity”, “biological activity of TRASH” or “functional activity of TRASH”, refers to an activity exerted by a TRASH protein, polypeptide or nucleic acid molecule on a TRASH responsive cell as determined in vivo, or in vitro, according to standard techniques. In one embodiment, a TRASH activity is a direct activity, such as an association with a cell-surface protein (e.g., a TRASH receptor). In another embodiment, a TRASH activity is an indirect activity, such as the induction of synthesis of a second protein (e.g. a cellular cytokine) mediated by interaction of the TRASH protein with a cell surface protein. In a preferred embodiment, a TRASH activity is at least one or more of the following activities: (i) interaction of a TRASH protein in the extracellular milieu with a non-TRASH protein molecule on the surface of the same cell which secreted the TRASH protein molecule; (ii) interaction of a TRASH protein in the extracellular milieu with a non-TRASH protein molecule on the surface of a different cell from that which secreted the TRASH protein molecule; (iii) complex formation between a TRASH protein and a TRASH receptor; (iv) complex formation between a TRASH protein and non-TRASH receptor; and (v) interaction of a TRASH protein with a second protein in the extracellular milieu. In yet another preferred embodiment, a TRASH activity is at least one or more of the following activities: (i) activation of a TRASH-dependent signal transduction pathway; (ii) cytolysis of certain tumor cell lines; (iii) modulation of secretion of inflammatory mediators/cytokines; (iv) modulation of the development or differentiation of a TRASH-expressing cell; (v) modulation of the development or differentiation of a non-TRASH-expressing cell; (vi) modulation of host resistance to infectious agents.

Accordingly, another embodiment of the invention features isolated TRASH proteins and polypeptides having a TRASH activity. Preferred TRASH proteins have a TNF signature motif and a TRASH activity. In another embodiment, the TRASH protein has a TNF-like N-terminal signal transmembrane anchor for a type II membrane protein and a TRASH activity. In another embodiment of the invention, the TRASH protein has a TNF signature motif, a TNF-like N-terminal signal transmembrane anchor for a type II membrane protein, and a TRASH activity. In another preferred embodiment, the TRASH protein has a TNF signature motif, a TNF-like N-terminal signal transmembrane anchor for a type II membrane protein, a TRASH activity, and an amino acid sequence sufficiently homologous to an amino acid sequence of SEQ ID NO:2, SEQ ID NO:7, SEQ ID NO:9, or SEQ ID NO:11. In still another preferred embodiment, the TRASH protein further comprises two cysteine residues that may be disulphide linked. In yet another preferred embodiment, the TRASH protein further comprises two putative N-linked glycosylation sites.

In a particularly preferred embodiment, the TRASH protein and nucleic acid molecules of the present invention are human TRASH molecules. A nucleotide sequence of an isolated human TRASH cDNA and the predicted amino acid sequence of the human TRASH protein are shown in FIG. 1 and in SEQ ID NO:1 and 2, respectively. In addition, the nucleotide sequence corresponding to the coding region of the human TRASH cDNA is represented as SEQ ID NO:3.

TRASH mRNA transcripts of approximately 1.5 kb were predominantly expressed in peripheral blood leukocytes. A transcript of approximately 1.5 kb is seen in the spleen and lymph nodes and lung. A transcript of approximately 1.7 kb is also observed in the colon, kidney, and spleen (See Example 2).

The human TRASH I cDNA set forth in SEQ ID NO:1, is approximately 1344 nucleotides in length and encodes a protein which is approximately 250 amino acid residues in length (SEQ ID NO:2). In a preferred embodiment, the human TRASH protein contains a TNF signature motif, a TNF-like N-terminal signal transmembrane anchor for a type II membrane protein, two cysteine residues that may be disulphide linked, and two putative N-linked glycosylation sites. A TRASH TNF signature motif can be found at least, for example, from about amino acids 155-171 of SEQ ID NO:2. A TRASH TNF-like N-terminal signal transmembrane anchor for a type II membrane protein can be found at least, for example, from about amino acids 1-44 of SEQ ID NO:2. Two cysteine residues that may be disulphide linked can be found at least, for example, at about amino acids 196 and 211 of SEQ ID. NO:2. Two putative N-linked glycosylation sites can be found at least, for example, at about amino acids 124 and 237 of SEQ ID NO:2.

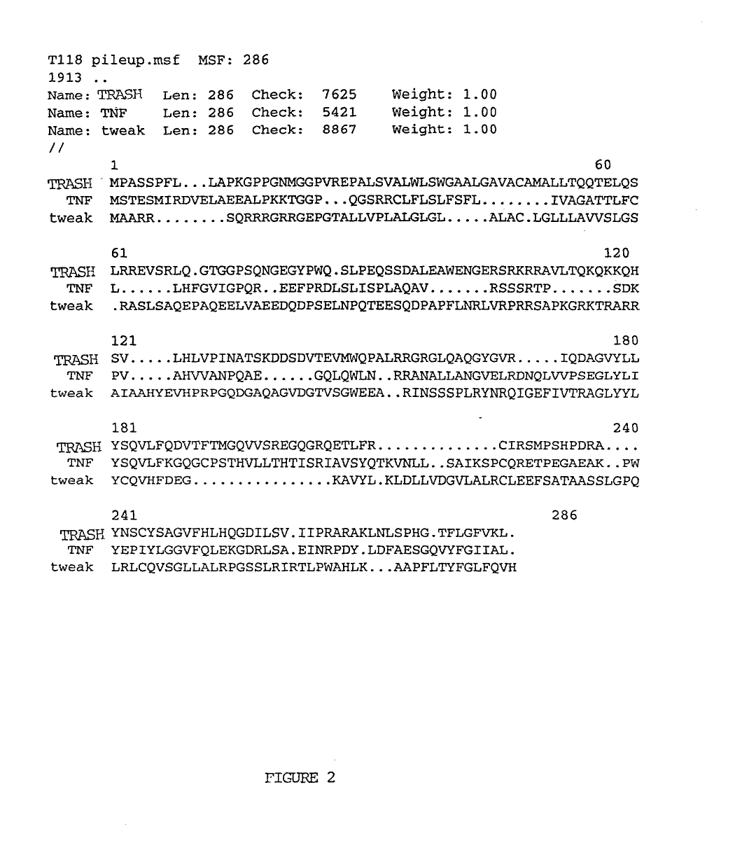

An alignment of the amino acid sequences of human TRASH (corresponding to amino acids 1-250 of SEQ ID NO:2), TNF-α (Swiss-Prot™ Accession No. PD01375), and human Tweak (Swiss-Prot™ Accession No. AF030099) is shown in FIG. 2. (The alignment was generated using MegAlign™ sequence alignment software. The initial pairwise alignment step was performed using a Wilbur-Lipmann algorithm with a K-tuple of 1, a GAP penalty of 3, a window of 5, and diagonals saved set to =5. The multiple alignment step was performed using the Clustal algorithm with a PAM 250 residue weight Table, a GAP penalty of 10, and a GAP length penalty of 10.)

Various aspects of the invention are described in further detail in the following subsections:

I. Isolated Nucleic Acid Molecules

One aspect of the invention pertains to isolated nucleic acid molecules that encode TRASH proteins or biologically active portions thereof, as well as nucleic acid fragments sufficient for use as hybridization probes to identify TRASH-encoding nucleic acids (e.g., TRASH mRNA) and fragments for use as PCR primers for the amplification or mutation of TRASH nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated TRASH nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, SEQ ID NO:3, SEQ ID NO:6, SEQ ID NO:8, SEQ ID NO:10, or a portion thereof, can be isolated using standard molecular biology techniques and the sequence information provided herein. Using all or portion of the nucleic acid sequence of SEQ ID NO:1, SEQ ID NO:3, SEQ ID NO:6, SEQ ID NO:8, or SEQ ID NO:10, as a hybridization probe, TRASH nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, SEQ ID NO:3, SEQ ID NO:6, SEQ ID NO:8, or SEQ ID NO:10 can be isolated by the polymerase chain reaction (PCR) using synthetic oligonucleotide primers designed based upon the sequence of SEQ ID NO:1, SEQ ID NO:3, SEQ ID NO:6, SEQ ID NO:8, or SEQ ID NO:10.

A nucleic acid of the invention can be amplified using cDNA, mRNA or alternatively, genomic DNA, as a template and appropriate oligonucleotide primers according to standard PCR amplification techniques. The nucleic acid so amplified can be closed into an appropriate vector and characterized by DNA sequence analysis. Furthermore, oligonucleotides corresponding to TRASH nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO:1, SEQ ID NO:3, SEQ ID NO:6, SEQ ID NO:8, SEQ ID NO:10. The sequence of SEQ ID NO:1 corresponds to a human TRASH cDNA. This cDNA comprises sequences encoding a human TRASH protein (i.e., “the coding region”, from nucleotides 273 to 1025), as well as 5′ untranslated sequences (nucleotides 1 to 272) and 3′ untranslated sequences (nucleotides 1026 to 1344). Alternatively, the nucleic acid molecule can comprise only the coding region of SEQ ID NO:1 (e.g., nucleotides 273 to 1025, corresponding to nucleotides 1-753 of SEQ ID NO:3).

The human TRASH cDNA of SEQ ID NO:1 also contains additional in frame ATG codons at positions 324-326 and 408-410 of SEQ ID NO:1 encoding methionines at positions 18 and 46 of SEQ ID NO:2, respectively. Thus, in another embodiment, the nucleotide sequence of SEQ ID NO:1 comprises a coding region from nucleotides 324 to 1025 (SEQ ID NO:8), as well as 5′ untranslated sequences from nucleotides 1 to 323 and 3′ untranslated sequences from nucleotides 1026 to 1344. In yet another embodiment, the nucleotide sequence of SEQ ID NO:1 comprises a coding region from nucleotides 408 to 1025 (SEQ ID NO:10), as well as 5′ untranslated sequences from nucleotides 1 to 407 and 3′ untranslated sequences from nucleotides 1026 to 1344.

In another preferred embodiment, an isolated nucleic acid molecule of the invention comprises a nucleic acid molecule which is a complement of the nucleotide sequence shown in SEQ ID NO:1, SEQ ID NO:3, SEQ ID NO:6, SEQ ID NO:8, SEQ ID NO:10, or a portion of any of these nucleotide sequences. A nucleic acid molecule which is complementary to the nucleotide sequence shown in SEQ ID NO:1, SEQ ID NO:3, SEQ ID NO:6, SEQ ID NO:8, or SEQ ID NO:10 is one which is sufficiently complementary to the nucleotide sequence shown in SEQ ID NO:1, SEQ ID NO:3, SEQ ID NO:6, SEQ ID NO:8, or SEQ ID NO:10 such that it can hybridize to the nucleotide sequence shown in SEQ ID NO:1, SEQ ID NO:3, SEQ ID NO:6, SEQ ID NO:8, or SEQ ID NO:10 thereby forming a stable duplex.

In still another preferred embodiment, an isolated nucleic acid molecule of the present invention comprises a nucleotide sequence which is at least about 60-65%, preferably at least about 70-75%, more preferable at least about 80-85%, and even more preferably at least about 90-95% or more homologous to the nucleotide sequence shown in SEQ ID NO:1, SEQ ID NO:3, SEQ ID NO:6, SEQ ID NO:8, SEQ ID NO:10, or a portion of any of these nucleotide sequences.

Moreover, the nucleic acid molecule of the invention can comprise only a portion of the nucleic acid sequence of SEQ ID NO:1 (or SEQ ID NO:3, SEQ ID NO:6, SEQ ID NO:8, SEQ ID NO:10) for example a fragment which can be used as a probe or primer or a fragment encoding a biologically active portion of TRASH. The nucleotide sequence determined from the cloning of the human TRASH genes allows for the generation of probes and primers designed for use in identifying and/or cloning TRASH homologues in other cell types, e.g., from other tissues, as well as TRASH homologues from other mammals. The probe/primer typically comprises substantially purified oligonucleotide. The oligonucleotide typically comprises a region of nucleotide sequence that hybridizes under stringent conditions to at least about 6, preferably about 25, more preferably about 40, 50, 100, 200, 300, 400, or 500 consecutive nucleotides of SEQ ID NO:1, SEQ ID NO:3, SEQ ID NO:6, SEQ ID NO:8, or SEQ ID NO:10 sense, or an anti-sense sequence of SEQ ID NO:1, SEQ ID NO:3, SEQ ID NO:6, SEQ ID NO:8, SEQ ID NO:10, or of a naturally occurring mutant of SEQ ID NO:1, SEQ ID NO:3, SEQ ID NO:6, SEQ ID NO:8, or SEQ ID NO:10.

Probes based on the human TRASH nucleotide sequence can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a TRASH protein, such as by measuring a level of a TRASH-encoding nucleic acid in a sample of cells from a subject e.g., detecting TRASH mRNA levels or determining whether a genomic TRASH gene has been mutated or deleted.

A nucleic acid fragment encoding a “biologically active portion of TRASH” can be prepared by isolating a portion of SEQ ID NO:1, SEQ ID NO:3, SEQ ID NO:6, SEQ ID NO:8, or SEQ ID NO:10 which encodes a polypeptide having a TRASH biological activity (the biological activities of the TRASH proteins have previously been described), expressing the encoded portion of TRASH protein (e.g., by recombinant expression in vitro) and assessing the activity of the encoded portion of TRASH.

In a preferred embodiment, probes based on the TRASH nucleotide sequence comprise nucleotides 1-503 or nucleotides 1338-1344 of SEQ ID NO:1. In another preferred embodiment, probes based on the TRASH nucleotide sequence comprise nucleotides 273-503 of SEQ ID NO: 1. In still another preferred embodiment, probes based on the TRASH nucleotide sequence comprise nucleotides 273-1344 or nucleotides 1-126 of SEQ ID NO:1.

The invention further encompasses nucleic acid molecules that differ from the nucleotide sequence shown in SEQ ID NO:1, (and portions thereof, e.g., SEQ ID NO:3, SEQ ID NO:6, SEQ ID NO:8, SEQ ID NO:10), or portions thereof, due to degeneracy of the genetic code and thus encode the same TRASH protein as that encoded by the nucleotide sequence shown in SEQ ID NO:1 (or SEQ ID NO:3, SEQ ID NO:6, SEQ ID NO:8, SEQ ID NO:10). In another embodiment, an isolated nucleic acid molecule of the invention has a nucleotide sequence encoding a protein having an amino acid sequence shown in SEQ ID NO:2, SEQ ID NO:7, SEQ ID NO:9, or SEQ ID NO:11.

In addition to the human TRASH nucleotide sequence shown in SEQ ID NO:1, SEQ ID NO:3, SEQ ID NO:6, SEQ ID NO:8, SEQ ID NO:10, it will be appreciated by those skilled in the art that DNA sequence polymorphisms that lead to changes in the amino acid sequences of TRASH may exist within a population (e.g., the human population). Such genetic polymorphism in the TRASH gene may exist among individuals within a population due to natural allelic variation. As used herein, the terms “gene” and “recombinant gene” refer to nucleic acid molecules comprising an open reading frame encoding a TRASH protein, preferably a mammalia TRASH protein. Such natural allelic variations can typically result in 1-5% variance in the nucleotide sequence of the TRASH gene. Any and all such nucleotide variations and resulting amino acid polymorphisms in TRASH that are the result of natural allelic variation and that do not alter the functional activity of TRASH are intended to be within the scope of the invention.

Moreover, nucleic acid molecules encoding TRASH proteins from other species, and thus which have a nucleotide sequence which differs from the human sequences of SEQ ID NO:1, SEQ ID NO:3, SEQ ID NO:6, SEQ ID NO:8, or SEQ ID NO:10 are intended to be within the scope of the invention. Nucleic acid molecules corresponding to natural allelic variants and homologues of the TRASH cDNA of the invention can be isolated based on their homology to the human or murine TRASH nucleic acids disclosed herein using the human cDNA, murine cDNA, or portions thereof, as a hybridization probe according to standard hybridization techniques under stringent hybridization conditions. For example, a viral TRASH cDNA can be isolated based on its homology to human or murine TRASH.

Accordingly, in another embodiment, an isolated nucleic acid molecule of the invention is at least 15 nucleotides in length and hybridizes under stringent conditions to the nucleic acid molecule comprising the nucleotide sequence of SEQ ID NO:1, SEQ ID NO:3, SEQ ID NO:6, SEQ ID NO:8, or SEQ ID NO:10. In other embodiment, the nucleic acid is at least 30, 50, 100, 250 or 500 nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 70%, more preferably at least about 80%, and even more preferably at least about 85 or 90% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6×sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of SEQ ID NO:1, SEQ ID NO:3, SEQ ID NO:6, SEQ ID NO:8, or SEQ ID NO:10, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the TRASH sequence that may exist in the population, the skilled artisan will further appreciate that changes can be introduced by mutation into the nucleotide sequence of SEQ ID NO:1, SEQ ID NO:3, SEQ ID NO:6, SEQ ID NO:8, or SEQ ID NO:10, thereby leading to changes in the amino acid sequence of the encoded TRASH protein, without altering the functional ability of the TRASH protein. For example, nucleotide substitutions leading to amino acid substitutions at “non-essential” amino acid residues can be made in the sequence of SEQ ID NO:1, SEQ ID NO:3, SEQ ID NO:6, SEQ ID NO:8, or SEQ ID NO:10. A “non-essential” amino acid residue is a residue that can be altered from the wild-type sequence of TRASH (e.g., the sequence of SEQ ID NO:2) without altering the biological activity, whereas an “essential” amino acid residue is required for biological activity. For example, amino acid residues that are conserved among the TRASH proteins of the present invention, are predicted to be particularly unamenable to alteration. Furthermore, amino acid residues that are conserved between TRASH protein and other proteins having TNFL-like domains are not likely to be amenable to alteration.

Accordingly, another aspect of the invention pertains to nucleic acid molecules encoding TRASH proteins that contain changes in amino acid residues that are not essential for activity. Such TRASH proteins differ in amino acid sequence from SEQ ID NO:2, SEQ ID NO:7, SEQ ID NO:9, or SEQ ID NO:11 yet retain biological activity. In one embodiment, the isolated nucleic acid molecule comprises a nucleotide sequence encoding a protein, wherein the protein comprises an amino acid sequence at least about 60% homologous to the amino acid sequence of SEQ ID NO:2, SEQ ID NO:7, SEQ ID NO:9, or SEQ ID NO:11. Preferably, the protein encoded by the nucleic acid molecule is at least about 65-70% homologous to SEQ ID NO:2, SEQ ID NO:7, SEQ ID NO:9, or SEQ ID NO:11, more preferably at least about 77-80% homologous to SEQ ID NO:2, SEQ ID NO:7, SEQ ID NO:9, or SEQ ID NO:11, even more preferably at least about 85-90% homologous to SEQ ID NO:2, SEQ ID NO:7, SEQ ID NO:9, or SEQ ID NO:11, and most preferably at least about 95% homologous to SEQ ID NO:2, SEQ ID NO:7, SEQ ID NO:9, or SEQ ID NO:11.

An isolated nucleic acid molecule encoding a TRASH protein homologous to the protein of SEQ ID NO:2, SEQ ID NO:7, SEQ ID NO:9, or SEQ ID NO:11 can be created by introducing one or more nucleotide substitutions, additions or deletions into the nucleotide sequence of SEQ ID NO:1 (or of SEQ ID NO:3, SEQ ID NO:6, SEQ ID NO:8, SEQ ID NO:10), such that one or more amino acid substitutions, additions or deletions are introduced into the encoded protein. Mutations can be introduced into SEQ ID NO:1, SEQ ID NO:3, SEQ ID NO:6, SEQ ID NO:8, or SEQ ID NO:10, by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in TRASH is preferably replaced with another amino acid residue from the same side chain family. Alternatively, in another embodiment, mutations can be introduced randomly along all or part of a TRASH coding sequence, such as by saturation mutagenesis, and the resultant mutants can be screened for TRASH biological activity to identify mutants that retain activity. Following mutagenesis of SEQ ID NO:1, SEQ ID NO:3, SEQ ID NO:6, SEQ ID NO:8, or SEQ ID NO:10, the encoded protein can be expressed recombinantly and the activity of the protein can be determined.

In a preferred embodiment, a mutant TRASH protein can be assayed for (1) activation of a TRASH-dependent signal transduction pathway; (2) cytolysis of certain tumor cell lines; (3) modulation of secretion of inflammatory mediators/cytokines; (4) modulation of the development or differentiation of a TRASH-expressing cell; (5) modulation of the development or differentiation of a non- TRASH-expressing cell; and (6) modulation of host resistance to infectious agents.

In addition to the nucleic acid molecules encoding TRASH proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire TRASH coding strand, or to only a portion thereof. In one embodiment, an antisense nucleic acid molecule is antisense to a “coding region” of the coding strand of a nucleotide sequence encoding TRASH. The term “coding region” refers to the region of the nucleotide sequence comprising codons which are translated into amino acid residues (e.g., the coding region of human TRASH corresponds to SEQ ID NO:3). In another embodiment, the antisense nucleic acid molecule is antisense to a “noncoding region” of the coding strand of a nucleotide sequence encoding TRASH. The term “noncoding region” refers to 5′ and 3′ sequences which flank the coding region that are not translated into amino acids (i.e., also referred to as 5′ and 3′ untranslated regions).

Given the coding strand sequences encoding TRASH disclosed herein (e.g., SEQ ID NO:3), antisense nucleic acids of the invention can be designed according to the rules of Watson and Crick base pairing. The antisense nucleic acid molecule can be complementary to the entire coding region of TRASH mRNA, but more preferably is an oligonucleotide which is antisense to only a portion of the coding or noncoding region of TRASH mRNA. For example, the antisense oligonucleotide can be complementary to the region surrounding the translation start site of TRASH mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a TRASH protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave TRASH mRNA transcripts to thereby inhibit translation of TRASH mRNA. A ribozyme having specificity for a TRASH-encoding nucleic acid can be designed based upon the nucleotide sequence of a TRASH cDNA disclosed herein i.e., SEQ ID NO:1, SEQ ID NO:3, SEQ ID NO:6, SEQ ID NO:8, or SEQ ID NO:10). For example, a derivative of a Tetrahymena L-19 IVS RNA can be constructed in which the nucleotide sequence of the active site is complementary to the nucleotide sequence to be cleaved in a TRASH-encoding mRNA. See, e.g., Cech et al. U.S. Pat. No. 4,987,071; and Cech et al. U.S. Pat. No. 5,116,742. Alternatively, TRASH mRNA can be used to select a catalytic RNA having a specific ribonuclease activity from a pool of RNA molecules. See, e.g., Bartel, D. and Szostak, J. W. (1993) Science 261:1411-1418.

Alternatively, TRASH gene expression can be inhibited by targeting nucleotide sequences complementary to the regulatory region of the TRASH (e.g., the TRASH promoter and/or enhancers) to form triple helical structures that prevent transcription of the TRASH gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In preferred embodiments, the nucleic acids of TRASH can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. PNAS 93:14670-675.

PNAs of TRASH can be used therapeutic and diagnostic applications. For example, PNAs can be used as antisense or antigene agents for sequence-specific modulation of gene expression by, e.g., inducing transcription or translation arrest or inhibiting replication. PNAs of TRASH can also be used, e.g., in the analysis of single base pair mutations in a gene by, e.g., PNA directed PCR clamping; as 'artificial restriction enzymes when used in combination with other enzymes, e.g., S1 nucleases (Hyrup B. (1996) supra); or as probes or primers for DNA sequence and hybridization (Hyrup B. et al. (1996) supra; Perry-O'Keefe supra).

In another embodiment, PNAs of TRASH can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of TRASH can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Research 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5:1119-11124).

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810, published Dec. 15, 1988) or the blood-brain barrier (see, e.g., PCT Publication No. W089/10134, published Apr. 25, 1988). In addition, oligonucleotides can be modified with hybridization-triggered cleavage agents (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

II. Isolated TRASH Proteins and Anti-TRASH Antibodies

One aspect of the invention pertains to isolated TRASH proteins, and biologically active portions thereof, as well as polypeptide fragments suitable for use as immunogens to raise anti-TRASH antibodies. In one embodiment, native TRASH proteins can be isolated from cells or tissue sources by an appropriate purification scheme using standard protein purification techniques. In another embodiment, TRASH proteins are produced by recombinant DNA techniques. Alternative to recombinant expression, a TRASH protein or polypeptide can be synthesized chemically using standard peptide synthesis techniques.

An “isolated” or “purified” protein or biologically active portion thereof is substantially free of cellular material or other contaminating proteins from the cell or tissue source from which the TRASH protein is derived, or substantially free from chemical precursors or other chemicals when chemically synthesized. The language “substantially free of cellular material” includes preparations of TRASH protein in which the protein is separated from cellular components of the cells from which it is isolated or recombinantly produced. In one embodiment, the language “substantially free of cellular material” includes preparations of TRASH protein having less than about 30% (by dry weight) of non-TRASH protein (also referred to herein as a “contaminating protein”), more preferably less than about 20% of non-TRASH protein, still more preferably less than about 10% of non-TRASH protein, and most preferably less than about 5% non-TRASH protein. When the TRASH protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of TRASH protein in which the protein is separated from chemical precursors or other chemicals which are involved in the synthesis of the protein. In one embodiment, the language “substantially free of chemical precursors or other chemicals” includes preparations of TRASH protein having less than about 30% (by dry weight) of chemical precursors or non-TRASH chemicals, more preferably less than about 20% chemical precursors or non-TRASH chemicals, still more preferably less than about 10% chemical precursors or non-TRASH chemicals, and most preferably less than about 5% chemical precursors or non-TRASH chemicals.

Biologically active portions of a TRASH protein include peptides comprising amino acid sequences sufficiently homologous to or derived from the amino acid sequence of the TRASH protein, e.g., the amino acid sequence shown in SEQ ID NO:2, which include less amino acids than the fill length TRASH proteins, and exhibit at least one activity of a TRASH protein. Typically, biologically active portions comprise a domain or motif with at least one activity of the TRASH protein. A biologically active portion of a TRASH protein can be a polypeptide which is, for example, 10, 25, 50, 100 or more amino acids in length.

In one embodiment, a biologically active portion of a TRASH protein comprises at least a TNF signature motif. In yet another embodiment, a biologically active portion of a TRASH protein comprises at least a TNF-like N-terminal signal transmembrane anchor for a type II membrane protein. In another embodiment, a biologically active portion of a TRASH protein further comprises two cysteine residues which may be disulphide linked. In another embodiment, a biologically active portion of a TRASH protein further comprises two putative N-linked glycosylation sites.

It is to be understood that a preferred biologically active portion of a TRASH protein of the present invention may contain at least one of the above-identified structural domains. Another preferred biologically active portion of a TRASH protein may contain at least two of the above-identified structural domains. Another preferred biologically active portion of a TRASH protein may contain at least three or more of the above-identified structural domains.

Moreover, other biologically active portions, in which other regions of the protein are deleted, can be prepared by recombinant techniques and evaluated for one or more of the functional activities of a native TRASH protein.

In a preferred embodiment, the TRASH protein has an amino acid sequence shown in SEQ ID NO:2, SEQ ID NO:7, SEQ ID NO:9, or SEQ ID NO:11. In other embodiments, the TRASH protein is substantially homologous to SEQ ID NO:2, SEQ ID NO:7, SEQ ID NO:9, or SEQ ID NO:11 and retains the functional activity of the protein of SEQ ID NO:2, SEQ ID NO:7, SEQ ID NO:9, or SEQ ID NO:11 differs in amino acid sequence due to natural allelic variation or mutagenesis, as described in detail in subsection I above. Accordingly, in another embodiment, the TRASH protein is a protein which comprises an amino acid sequence at least about 60% homologous to the amino acid sequence of SEQ ID NO:2, SEQ ID NO:7, SEQ ID NO:9, or SEQ ID NO:11 and, preferably, retains a functional activity of the TRASH proteins of SEQ ID NO:2, SEQ ID NO:7, SEQ ID NO:9, or SEQ ID NO:11. Preferably, the protein is at least about 70% homologous to SEQ ID NO:2, SEQ ID NO:7, SEQ ID NO:9, or SEQ ID NO:11, more preferably at least about 80% homologous to SEQ ID NO:2, SEQ ID NO:7, SEQ ID NO:9, or SEQ ID NO:11, even more preferably at least about 90% homologous to SEQ ID NO:2, SEQ ID NO:7, SEQ ID NO:9, or SEQ ID NO:11, and most preferably at least about 95% or more homologous to SEQ ID NO:2, SEQ ID NO:7, SEQ ID NO:9, or SEQ ID NO:11.

To determine the percent homology of two amino acid sequences or of two nucleic acids, the sequences are aligned for optimal comparison purposes (e.g., gaps can be introduced in the sequence of a first amino acid or nucleotide sequence for optimal alignment with a second amino acid or nucleotide sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100). The determination of percent homology between two sequences can be accomplished using a mathematical algorithm. A preferred, non-limiting example of a mathematical algorithim utilized for the comparison of two sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs of Altschul, et al. (1990) J. Mol. Biol. 215:403-10. BLAST nucleotide searches can be performed performed with the NBLAST program, score=100, wordlength=12 to obtain nucleotide sequences homologous to CRSP nucleic acid molecules of the invention. BLAST protein searches can be performed with the XBLAST program, score=50, wordlength=3 to obtain amino acid sequences homologous to CRSP protein molecules of the invention. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithim utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The invention also provides TRASH chimeric or fusion proteins. As used herein, a TRASH “chimeric protein” or “fusion protein” comprises a TRASH polypeptide operatively linked to a non-TRASH polypeptide. A “TRASH polypeptide” refers to a polypeptide having an amino acid sequence corresponding to TRASH, whereas a “non-TRASH polypeptide” refers to a polypeptide having an amino acid sequence corresponding to a protein which is not substantially homologous to the TRASH protein, e.g., a protein which is different from the TRASH protein and which is derived from the same or a different organism. Within a TRASH fusion protein the TRASH polypeptide can correspond to all or a portion of a TRASH protein. In a preferred embodiment, a TRASH fusion protein comprises at least one biologically active portion of a TRASH protein. In another preferred embodiment, a TRASH fusion protein comprises at least two biologically active portions of a TRASH protein. In another preferred embodiment, a TRASH fusion protein comprises at least three biologically active portions of a TRASH protein. Within the fusion protein, the term “operatively linked” is intended to indicate that the TRASH polypeptide and the non-TRASH polypeptide are fused in-frame to each other. The non-TRASH polypeptide can be fused to the N-terminus or C-terminus of the TRASH polypeptide.

For example, in one embodiment, the fusion protein is a GST-TRASH fusion protein in which the TRASH sequences are fused to the C-terminus of the GST sequences. Such fusion proteins can facilitate the purification of recombinant TRASH.

In another embodiment, the fusion protein is a TRASH protein containing a heterologous signal sequence at its N-terminus. For example, the native TRASH TNF-like N-terminal signal transmembrane anchor for a type II membrane protein sequence (i.e, about amino acids 1 to 44 of SEQ ID NO:2) can be removed and replaced with a signal sequence from another protein. In certain host cells (e.g., mammalian host cells), expression and/or secretion of TRASH can be increased through use of a heterologous signal sequence.

In yet another embodiment, the fusion protein is a TRASH-immunoglobulin fusion protein in which the TRASH sequences comprising primarily the TRASH extracellular domain are fused to sequences derived from a member of the immunoglobulin protein family. Soluble derivatives have also been made of cell surface glycoproteins in the immunoglobulin gene superfamily consisting of an extracellular domain of the cell surface glycoprotein fused to an immunoglobulin constant (Fc) region (see e.g., Capon, D. J. et al. (1989) Nature 337:525-531 and Capon U.S. Pat. Nos. 5,116,964 and 5,428,130 [CD4-IgG1 constructs]; Linsley, P. S. et al. (1991) J. Exp. Med. 173:721-730 [a CD28-IgG1 construct and a B7-1-IgG1 construct]; and Linsley, P. S. et al. (1991) J. Exp. Med. 174:561-569 and U.S. Pat. No. 5,434,131 [a CTLA4-IgG1]). Such fusion proteins have proven useful for modulating receptor-ligand interactions. Soluble derivatives of cell surface proteins of the tumor necrosis factor receptor (TNFR) superfamily proteins have been made consisting of an extracellular domain of the cell surface receptor fused to an immunoglobulin constant (Fc) region (see for example Moreland et al. (1997) N. Engl. J. Med. 337(3):141-147; van der Poll et al. (1997) Blood 89(10):3727-3734; and Ammann et al. (1997) J. Clin. Invest. 99(7):1699-1703.) Furthermore, fusion proteins have been made using the CH2 and CH3 domains of IgG fused downstream of murine IL-17 leader sequences and upstream of murine CTLA-8 sequences and upstream of HVS13 sequences (see for example Yao et al. (1995) Immunity 8:811-821.)

The TRASH-immunoglobulin fusion proteins of the invention can be incorporated into pharmaceutical compositions and administered to a subject to inhibit an interaction between a TRASH protein and a TRASH receptor on the surface of a cell, to thereby suppress TRASH-mediated cellular function in vivo. The TRASH-immunoglobulin fusion proteins can be used to affect the bioavailability of a TRASH protein. Inhibition of the TRASH protein/TRASH receptor interaction may be useful therapeutically, for example, in regulation of the cellular immune response, regulation of inflammation, or regulation of hematopoiesis. Moreover, the TRASH-immunoglobulin fusion proteins of the invention can be used as immunogens to produce anti-TRASH antibodies in a subject, to purify TRASH receptors and in screening assays to identify molecules which inhibit the interaction of a TRASH protein with a TRASH receptor.

Preferably, a TRASH chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A TRASH-encoding nucleic acid can be cloned into such an expression vector such that the fusion moiety is linked in-frame to the TRASH protein.

The present invention also pertains to variants of the TRASH proteins which function as either TRASH agonists (mimetics) or as TRASH antagonists. Variants of the TRASH protein can be generated by mutagenesis, e.g., discrete point mutation or truncation of the TRASH protein. An agonist of the TRASH protein can retain substantially the same, or a subset, of the biological activities of the naturally occurring form of the TRASH protein. An antagonist of the TRASH protein can inhibit one or more of the activities of the naturally occurring form of the TRASH protein by, for example, competitively binding to a TRASH receptor of TRASH-binding protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the TRASH proteins.

In one embodiment, variants of the TRASH protein which function as either TRASH agonists (mimetics) or as TRASH antagonists can be identified by screening combinatorial libraries of mutants, (e.g., truncation mutants) of the TRASH protein for TRASH protein agonist or antagonist activity. In one embodiment, a variegated library of TRASH variants is generated by combinatorial mutagenesis at the nucleic acid level and is encoded by a variegated gene library. A variegated library of TRASH variants can be produced by, for example, enzymatically ligating a mixture of synthetic oligonucleotides into gene sequences such that a degenerate set of potential TRASH sequences is expressible as individual polypeptides, or alternatively, as a set of larger fusion proteins (e.g., for phage display) containing the set of TRASH sequences therein. There are a variety of methods which can be used to produce libraries of potential TRASH variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential TRASH sequences. Methods for synthesizing degenerate oligonucleotides are known in the art (see, e.g., Narang, S. A. (1983) Tetrahedron 39:3; Itakura et al. (1984) i Annu. Rev. Biochem. 53:323; Itakura et al. (1984) Science 198:1056; Ike et al. (1983) Nucleic Acid Res. 11:477.

In addition, libraries of fragments of the TRASH protein coding sequence can be used to generate a variegated population of TRASH fragments for screening and subsequent selection of variants of a TRASH protein. In one embodiment, a library of coding sequence fragments can be generated by treating a double stranded PCR fragment of a TRASH coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the TRASH protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of TRASH proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique which enhances the frequency of functional mutants in the libraries, can be used in combination with the screening assays to identify TRASH variants (Arkin and Yourvan (1992) PNAS 89:7811-7815; Delgrave et al. (1993) Protein Engineering 6(3):327-331).

In one embodiment, cell based assays can be exploited to analyze a variegated TRASH library. For example, a library of expression vectors can be transfected into a cell line which ordinarily secretes TRASH protein. Supernatants from the transfected cells are then contacted with TRASH-responsive cells and the effect of the mutation in TRASH can be detected, e.g., by measuring any of a number of TRASH-responsive cell responses. Plasmid DNA can then be recovered from the mutant TRASH-secreting cells which score for inhibition, or alternatively, potentiation of the TRASH-dependent response, and the individual clones further characterized.

An isolated TRASH protein, or a portion or fragment thereof, can be used as an immunogen to generate antibodies that bind TRASH using standard techniques for polyclonal and monoclonal antibody preparation. The full-length TRASH protein can be used or, alternatively, the invention provides antigenic peptide fragments of TRASH for use as immunogens. The antigenic peptide of TRASH comprises at least 8 amino acid residues of the amino acid sequence shown in SEQ ID NO:2, SEQ ID NO:7, SEQ ID NO:9, or SEQ ID NO:11 and encompasses an epitope of TRASH such that an antibody raised against the peptide forms a specific immune complex with TRASH. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of TRASH that are located on the surface of the protein, e.g., hydrophilic regions.

A TRASH immunogen typically is used to prepare antibodies by immunizing a suitable subject, (e.g., rabbit, goat, mouse or other mammal) with the immunogen. An appropriate immunogenic preparation can contain, for example, recombinantly expressed TRASH protein or a chemically synthesized TRASH polypeptide. The preparation can further include an adjuvant, such as Freund's complete or incomplete adjuvant, or similar immunostimulatory agent. Immunization of a suitable subject with an immunogenic TRASH preparation induces a polyclonal anti-TRASH antibody response.

Accordingly, another aspect of the invention pertains to anti-TRASH antibodies. The term “antibody” as used herein refers to immunoglobulin molecules and immunologically active portions of immunoglobulin molecules, i.e., molecules that contain an antigen binding site which specifically binds (immunoreacts with) an antigen, such as TRASH. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind TRASH. The term “monoclonal antibody” or “monoclonal antibody composition”, as used herein, refers to a population of antibody molecules that contain only one species of an antigen binding site capable of immunoreacting with a particular epitope of TRASH. A monoclonal antibody composition thus typically displays a single binding affinity for a particular TRASH protein with which it immunoreacts.

Polyclonal anti-TRASH antibodies can be prepared as described above by immunizing a suitable subject with a TRASH immunogen. The anti-TRASH antibody titer in the immunized subject can be monitored over time by standard techniques, such as with an enzyme linked immunosorbent assay (ELISA) using immobilized TRASH. If desired, the antibody molecules directed against TRASH can be isolated from the mammal (e.g., from the blood) and further purified by well known techniques, such as protein A chromatography to obtain the IgG fraction. At an appropriate time after immunization, e.g., when the anti-TRASH antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lemer (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a TRASH immunogen as described above, and the culture supernatants of the resulting hybridoma cells are screened to identify a hybridoma producing a monoclonal antibody that binds TRASH.

Any of the many well known protocols used for fusing lymphocytes and immortalized cell lines can be applied for the purpose of generating an anti-TRASH monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind TRASH, e.g., using a standard ELISA assay.

Alternative to preparing monoclonal antibody-secreting hybridomas, a monoclonal anti-TRASH antibody can be identified and isolated by screening a recombinant combinatorial immunoglobulin library (e.g., an antibody phage display library) with TRASH to thereby isolate immunoglobulin library members that bind TRASH. Kits for generating and screening phage display libraries are commercially available (e.g., the Pharmacia Recombinant Phage Antibody System, Catalog No. 27-9400-0 1; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554.

Additionally, recombinant anti-TRASH antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-TRASH antibody (e.g., monoclonal antibody) can be used to isolate TRASH by standard techniques, such as affinity chromatography or immunoprecipitation. An anti-TRASH antibody can facilitate the purification of natural TRASH from cells and of recombinantly produced TRASH expressed in host cells. Moreover, an anti-TRASH antibody can be used to detect TRASH protein (e.g., in a cellular lysate or cell supernatant) in order to evaluate the abundance and pattern of expression of the TRASH protein. Anti-TRASH antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid encoding TRASH (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., TRASH proteins, mutant forms of TRASH, fusion proteins, etc.).

The recombinant expression vectors of the invention can be designed for expression of TRASH in prokaryotic or eukaryotic cells. For example, TRASH can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in TRASH activity assays, in TRASH ligand binding (e.g., direct assays or competitive assays described in detail below), to generate antibodies specific for TRASH proteins, as examples. In a preferred embodiment, a TRASH fusion expressed in a retroviral expression vector of the present invention can be utilized to infect bone marrow cells which are subsequently transplanted into irradiated recipients. The pathology of the subject recipient is then examined after sufficient time has passed (e.g six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21 (DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the TRASH expression vector is a yeast expression vector. Examples of vectors for expression in yeast S. cerivisae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, TRASH can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to TRASH mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, TRASH protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding TRASH or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) TRASH protein. Accordingly, the invention further provides methods for producing TRASH protein using the host cells of the invention. In one embodiment, the method comprises culturing the host cell of invention (into which a recombinant expression vector encoding TRASH has been introduced) in a suitable medium such that TRASH protein is produced. In another embodiment, the method further comprises isolating TRASH from the medium or the host cell.

The host cells of the invention can also be used to produce nonhuman transgenic animals. For example, in one embodiment, a host cell of the invention is a fertilized oocyte or an embryonic stem cell into which TRASH-coding sequences have been introduced. Such host cells can then be used to create non-human transgenic animals in which exogenous TRASH sequences have been introduced into their genome or homologous recombinant animals in which endogenous TRASH sequences have been altered. Such animals are useful for studying the function and/or activity of TRASH and for identifying and/or evaluating modulators of TRASH activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous TRASH gene has been altered by homologous recombination between the endogenous gene and an exogenous DNA molecule introduced into a cell of the animal, e.g., an embryonic cell of the animal, prior to development of the animal.

A transgenic animal of the invention can be created by introducing TRASH-encoding nucleic acid into the male pronuclei of a fertilized oocyte, e.g., by microinjection, retroviral infection, and allowing the oocyte to develop in a pseudopregnant female foster animal. The TRASH cDNA sequences of SEQ ID NO:1, SEQ ID NO:3, SEQ ID NO:6, SEQ ID NO:8, or SEQ ID NO:10 can be introduced as a transgene into the genome of a non-human animal. Intronic sequences and polyadenylation signals can also be included in the transgene to increase the efficiency of expression of the transgene. A tissue-specific regulatory sequence(s) can be operably linked to the TRASH transgene to direct expression of TRASH protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the TRASH transgene in its genome and/or expression of TRASH mRNA in tissues or cells of the animals. A transgenic founder animal can then be used to breed additional animals carrying the transgene. Moreover, transgenic animals carrying a transgene encoding TRASH can further be bred to other transgenic animals carrying other transgenes.

To create a homologous recombinant animal, a vector is prepared which contains at least a portion of a TRASH gene into which a deletion, addition or substitution has been introduced to thereby alter, e.g., functionally disrupt, the TRASH gene. The TRASH gene can be a human gene (e.g., the cDNA of SEQ ID NO:1), but more preferably, is a non-human homologue of a human TRASH gene. For example, a mouse TRASH gene can be used to construct a homologous recombination vector suitable for altering an endogenous TRASH gene in the mouse genome. In a preferred embodiment, the vector is designed such that, upon homologous recombination, the endogenous TRASH gene is functionally disrupted (i.e., no longer encodes a functional protein; also referred to as a “knock out” vector). Alternatively, the vector can be designed such that, upon homologous recombination, the endogenous TRASH gene is mutated or otherwise altered but still encodes functional protein (e.g., the upstream regulatory region can be altered to thereby alter the expression of the endogenous TRASH protein). In the homologous recombination vector, the altered portion of the TRASH gene is flanked at its 5′ and 3′ ends by additional nucleic acid of the TRASH gene to allow for homologous recombination to occur between the exogenous TRASH gene carried by the vector and an endogenous TRASH gene in an embryonic stem cell. The additional flanking TRASH nucleic acid is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced TRASH gene has homologously recombined with the endogenous TRASH gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The TRASH nucleic acid molecules, TRASH proteins, and anti-TRASH antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a TRASH protein or anti-TRASH antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) detection assays (e.g., chromosomal mapping, tissue typing, forensic biology), c) predictive medicine (e.g., diagnostic assays, prognostic assays, monitoring clinical trials); and d) methods of treatment (e.g., therapeutic and prophylactic methods as well as such methods in the context of pharmacogenomics). As described herein, a TRASH protein of the invention has one or more of the following activities: (i) activation of a TRASH-dependent signal transduction pathway; (ii) cytolysis of certain tumor cell lines; (iii) modulation of secretion of inflammatory mediators/cytokines; (iv) modulation of the development or differentiation of a TRASH-expressing cell; (v) modulation of the development or differentiation of a non- TRASH-expressing cell; or (vi) modulation of host resistance to infectious agents. The isolated nucleic acid molecules of the invention can be used, for example, to express TRASH protein (e.g., via a recombinant expression vector in a host cell in gene therapy applications), to detect TRASH mRNA (e.g., in a biological sample) or a genetic alteration in a TRASH gene, and to modulate TRASH activity, as described further below. In addition, the TRASH proteins can be used to screen drugs or compounds which modulate the TRASH activity as well as to treat disorders characterized by insufficient or excessive production of TRASH protein or production of TRASH protein forms which have decreased or aberrant activity compared to TRASH wild type protein (e.g., differentiative or developmental disorders). Moreover, soluble forms of the TRASH protein can be used to bind other membrane-bound cytokine receptors and influence bioavailability of such a receptors cognate ligand. In addition, the anti-TRASH antibodies of the invention can be used to detect and isolate TRASH proteins and modulate TRASH activity.

A. Screening Assays

The invention provides a method (also referred to herein as a “screening assay”) for identifying modulators, i.e., candidate or test compounds or agents (e.g., peptides, peptidomimetics, small molecules or other drugs) which bind to TRASH proteins or have a stimulatory or inhibitory effect on, for example, TRASH expression or TRASH activity.

In one embodiment, the invention provides assays for screening candidate or test compounds which bind to or modulate the activity of the membrane-bound form of a TRASH protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990)Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a membrane-bound form of TRASH protein, or a biologically active portion thereof, on the cell surface is contacted with a test compound and the ability of the test compound to bind to a TRASH protein determined. The cell, for example, can of mammalian origin or a yeast cell. Determining the ability of the test compound to bind to the TRASH protein can be accomplished, for example, by coupling the test compound with a radioisotope or enzymatic label such that binding of the test compound to the TRASH protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In a preferred embodiment, the assay comprises contacting a cell which expresses a membrane-bound form of TRASH protein, or a biologically active portion thereof, on the cell surface with a known compound which binds TRASH to form an assay mixture, contacting the assay mixture with a test compound, and determining the ability of the test compound to interact with a TRASH protein, wherein determining the ability of the test compound to interact with a TRASH protein comprises determining the ability of the test compound to preferentially bind to TRASH or a biologically active portion thereof as compared to the known compound.

In another embodiment, an assay is a cell-based assay comprising contacting a cell expressing a membrane-bound form of TRASH protein, or a biologically active portion thereof, on the cell surface with a test compound and determining the ability of the test compound to modulate (e.g. stimulate or inhibit) the activity of the TRASH protein or biologically active portion thereof. Determining the ability of the test compound to modulate the activity of TRASH or a biologically active portion thereof can be accomplished, for example, by determining the ability of the TRASH protein to bind to or interact with a TRASH target molecule. As used herein, a “target molecule” is a molecule with which a TRASH protein binds or interacts in nature, for example, a molecule on the surface of a cell which expresses a TRASH protein, a molecule on the surface of a second cell, a molecule in the extracellular milieu, a molecule associated with the internal surface of a cell membrane or a cytoplasmic molecule. A TRASH target molecule can be a non-TRASH molecule or a TRASH protein or polypeptide of the present invention. In one embodiment, a TRASH target molecule is a component of a signal transduction pathway which facilitates transduction of an extracellular signal (e.g. a signal generated by binding of a compound to a membrane-bound TRASH molecule) through the cell membrane and into the cell. The target, for example, can be a second intercellular protein which has catalytic activity or a protein which facilitates the association of downstream signaling molecules with TRASH. Alternatively, the target molecule can be a substrate for a catalytic activity of the TRASH protein.

Determining the ability of the TRASH protein to bind to or interact with a TRASH target molecule can be accomplished by one of the methods described above for determining direct binding. In a preferred embodiment, determining the ability of the TRASH protein to bind to or interact with a TRASH target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a TRASH-responsive regulatory element operatively linked to a nucleic acid encoding a detectable marker, e.g. luciferase), or detecting a cellular response, for example, cell survival, cellular differentiation, or cell proliferation.

In yet another embodiment, an assay of the present invention is a cell-free assay comprising contacting a TRASH protein or biologically active portion thereof with a test compound and determining the ability of the test compound to bind to the TRASH protein or biologically active portion thereof. Binding of the test compound to the TRASH protein can be determined either directly or indirectly as described above. In a preferred embodiment, the assay comprises contacting the TRASH protein or biologically active portion thereof with a known compound which binds TRASH to form an assay mixture, contacting the assay mixture with a test compound, and determining the ability of the test compound to interact with a TRASH protein, wherein determining the ability of the test compound to interact with a TRASH protein comprises determining the ability of the test compound to preferentially bind to TRASH or biologically active portion thereof as compared to the known compound.

In another embodiment, an assay is a cell-free assay comprising contacting TRASH protein or biologically active portion thereof with a test compound and determining the ability of the test compound to modulate (e.g. stimulate or inhibit) the activity of the TRASH protein or biologically active portion thereof. Determining the ability of the test compound to modulate the activity of TRASH can be accomplished, for example, by determining the ability of the TRASH protein to bind to a TRASH target molecule by one of the methods described above for determining direct binding. In an alternative embodiment, determining the ability of the test compound to modulate the activity of TRASH can be accomplished by determining the ability of the TRASH protein further modulate a TRASH target molecule. For example, the catalytic/enzymatic activity of the target molecule on an appropriate substrate can be determined as previously described.

In yet another embodiment, the cell-free assay comprises contacting the TRASH protein or biologically active portion thereof with a known compound which binds TRASH to form an assay mixture, contacting the assay mixture with a test compound, and determining the ability of the test compound to interact with a TRASH protein, wherein determining the ability of the test compound to interact with a TRASH protein comprises determining the ability of the TRASH protein to preferentially bind to or modulate the activity of a TRASH target molecule.

The cell-free assays of the present invention are amenable to use of both the soluble form or the membrane-bound form of TRASH. In the case of cell-free assays comprising the membrane-bound form of TRASH, it may be desirable to utilize a solubilizing such that the membrane-bound form of TRASH is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either TRASH or its target molecule to facilitate separation of complexed from uncomplexed forms of one or both of the proteins, as well as to accommodate automation of the assay. Binding of a test compound to TRASH, or interaction of TRASH with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/TRASH fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or TRASH protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of TRASH binding or activity determined using standard techniques.

Other techniques for immobilizing proteins on matrices can also be used in the screening assays of the invention. For example, either TRASH or its target molecule can be immobilized utilizing conjugation of biotin and streptavidin. Biotinylated TRASH or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with TRASH or target molecules but which do not interfere with binding of the TRASH protein to its target molecule can be derivatized to the wells of the plate, and unbound target or TRASH trapped in the wells by antibody conjugation. Methods for detecting such complexes, in addition to those described above for the GST-immobilized complexes, include immunodetection of complexes using antibodies reactive with the TRASH or target molecule, as well as enzyme-linked assays which rely on detecting an enzymatic activity associated with the TRASH or target molecule.

In another embodiment, modulators of TRASH expression are identified in a method wherein a cell is contacted with a candidate compound and the expression of TRASH mRNA or protein in the cell is determined. The level of expression of TRASH mRNA or protein in the presence of the candidate compound is compared to the level of expression of TRASH mRNA or protein in the absence of the candidate compound. The candidate compound can then be identified as a modulator of TRASH expression based on this comparison. For example, when expression of TRASH mRNA or protein is greater (statistically significantly greater) in the presence of the candidate compound than in its absence, the candidate compound is identified as a stimulator of TRASH mRNA or protein expression. Alternatively, when expression of TRASH mRNA or protein is less (statistically significantly less) in the presence of the candidate compound than in its absence, the candidate compound is identified as an inhibitor of TRASH mRNA or protein expression. The level of TRASH mRNA or protein expression in the cells can be determined by methods described herein for detecting TRASH mRNA or protein.

In yet another aspect of the invention, the TRASH proteins can be used as “bait proteins” in a two-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent W094/10300), to identify other proteins, which bind to or interact with TRASH (“TRASH-binding proteins” or “TRASH-bp”) and modulate TRASH activity. Such TRASH-binding proteins are also likely to be involved in the propagation of signals by the TRASH proteins as, for example, upstream or downstream elements of the TRASH pathway.

The two-hybrid system is based on the modular nature of most transcription factors, which consist of separable DNA-binding and activation domains. Briefly, the assay utilizes two different DNA constructs. In one construct, the gene that codes for TRASH is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a TRASH-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with TRASH.

This invention further pertains to novel agents identified by the above-described screening assays and uses thereof for treatments as described herein.

In another embodiment, the invention provides assays for screening candidate or test compounds which bind to or modulate the activity of a TRASH target molecule. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are compared to a control assay comprising cell expressing a TRASH target molecule, TRASH protein and IL-1 without the test compound.

Determining the ability of the TRASH protein to bind to or interact with a TRASH target molecule can be accomplished by determining the activity of the target molecule. For example, the activity of the target molecule can be determined by detecting induction or lack of induction of a cellular second messenger of the target (i.e. intracellular Ca²⁺, diacylglycerol, IP₃, PGE₂, etc.), detecting catalytic/enzymatic activity of the target an appropriate substrate, detecting the induction of a reporter gene (comprising a IL-1-responsive regulatory element operatively linked to a nucleic acid encoding a detectable marker, e.g., luciferase), or detecting a cellular response or lack of a cellular response, for example, IL-1 stimulated development, differentiation or the rate of IL-1 stimulated proliferation.

In yet another embodiment, the assay is a cell-free assay in which a TRASH protein or biologically active portion thereof is contacted with a test compound and the ability of the test compound to modulate (e.g., stimulate or inhibit) the activity of the TRASH protein or biologically active portion thereof is determined. Determining the ability of the test compound to modulate the activity of a TRASH protein can be accomplished, for example, by determining the ability of the TRASH protein to bind to a TRASH target molecule in the presence of IL-1 in the absence and presence of the test compound. Determining the ability of the TRASH protein to bind to a TRASH target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon surface plasmon resonance (SPR) can be used as an indication of real-time reactions between biological molecules.

In an alternative embodiment, determining the ability of the test compound to modulate the activity of a TRASH protein can be accomplished by determining the ability of the TRASH protein and a TRASH target molecule to further modulate the activity of IL-1. For example, the presence or absence of an IL-1 stimulated activity can be determined as previously described.

In more than one embodiment of the above assay methods of the present invention, it may be desirable to immobilize either TRASH or its target molecule to facilitate separation of complexed from uncomplexed forms of one or both of the proteins, as well as to accommodate automation of the assay. Binding of a test compound to a TRASH protein, or interaction of a TRASH protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/TRASH fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or TRASH protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of TRASH binding or activity determined using standard techniques.

Other techniques for immobilizing proteins on matrices can also be used in the screening assays of the invention. For example, either a TRASH protein or a TRASH target molecule can be immobilized utilizing conjugation of biotin and streptavidin. Biotinylated TRASH protein or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with TRASH protein or target molecules but which do not interfere with binding of the TRASH protein to its target molecule can be derivatized to the wells of the plate, and unbound target or TRASH protein trapped in the wells by antibody conjugation. Methods for detecting such complexes, in addition to those described above for the GST-immobilized complexes, include immunodetection of complexes using antibodies reactive with the TRASH protein or target molecule, as well as enzyme-linked assays which rely on detecting an enzymatic activity associated with the TRASH protein or target molecule.

In yet another aspect of the invention, the TRASH proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO094/10300), to identify other proteins, which bind to or interact with TRASH (“TRASH-binding proteins” or “TRASH-bp”) and modulate TRASH activity. Such TRASH-binding proteins are also likely to be involved in the propagation of signals by the TRASH proteins as, for example, downstream elements of a TRASH-mediated signaling pathway. Alternatively, such TRASH-binding proteins are likely to be cell-surface molecules associated with non-TRASH expressing cells, wherein such TRASH-binding proteins are involved in signal transduction.

The two-hybrid system is based on the modular nature of most transcription factors, which consist of separable DNA-binding and activation domains. Briefly, the assay utilizes two different DNA constructs. In one construct, the gene that codes for a TRASH protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a TRASH-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the TRASH protein.

This invention further pertains to novel TRASH agents such as TRASH proteins or biologically active portions thereof, TRASH variants which function as IL-1 agonists and nucleic acid molecules encoding a TRASH protein or variant, which can be screened to determine the efficacy of such agents on various IL-1 stimulated activities (e.g., IL-1 stimulated immune response, IL-1 stimulated proliferation, IL-1 stimulated transduction pathway, or IL-1 stimulated differentiation).

In one embodiment, determining the ability of a TRASH agent to modulate the activity of IL-1 can be accomplished by testing the ability of TRASH to interfere with the proliferation of T cells in the presence of IL-1.

It is also within the scope of this invention to further use a TRASH agent as described herein in an appropriate animal model. For example, an agent as described herein (e.g., an IL-1 modulating agent) can be used in an animal model to determine the efficacy, toxicity, or side effects of treatment with such an agent. Alternatively, a TRASH agent identified as described herein can be used in an animal model to determine the mechanism of action of such an agent. Animal models for use in determining the efficacy or mechanism of action of a TRASH agent of the present invention include animal models demonstrating parameters of sepsis (e.g., animals injected with E.coli to induce hypotension) and animal models for determining bone metabolism (e.g., lethally irradiated mice which have been transplanted with TRASH infected marrow cells). Other animal models which are recognized in the art as predictive of results in humans with various IL-1 induced disorders are known in the art and described, for example, in Dinarello (1991) Blood 77(8):1627-1652. Furthermore, this invention pertains to uses of TRASH agents and agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the TRASH, sequences, described herein, can be used to map the location of the TRASH genes, respectively, on a chromosome. The mapping of the TRASH sequences to chromosomes is an important first step in correlating these sequences with genes associated with disease.

Briefly, TRASH genes can be mapped to chromosomes by preparing PCR primers (preferably 15-25 bp in length) from the TRASH sequences. Computer analysis of the TRASH, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the TRASH sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the TRASH sequences to design oligonucleotide primers, sublocalization can be achieved with panels of fragments from specific chromosomes. Other mapping strategies which can similarly be used to map a 9o, 1p, or 1v sequence to its chromosome include in situ hybridization (described in Fan, Y. et al. (1990) PNAS, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the TRASH gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The TRASH sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the TRASH sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The TRASH sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1, SEQ ID NO:3, SEQ ID NO:6, SEQ ID NO:8, SEQ ID NO:10, can comfortably provide positive individual identification with a panel of perhaps 10 to 1,000 primers which each yield a noncoding amplified sequence of 100 bases. If predicted coding sequences, such as those in SEQ ID NO:3 are used, a more appropriate number of primers for positive individual identification would be 500-2,000.

If a panel of reagents from TRASH sequences described herein is used to generate a unique identification database for an individual, those same reagents can later be used to identify tissue from that individual. Using the unique identification database, positive identification of the individual, living or dead, can be made from extremely small tissue samples.

3. Use of Partial TRASH Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1, SEQ ID NO:3, SEQ ID NO:6, SEQ ID NO:8, SEQ ID NO:10, are particularly appropriate for this use as greater numbers of polymorphisms occur in the noncoding regions, making it easier to differentiate individuals using this technique. Examples of polynucleotide reagents include the TRASH sequences or portions thereof, e.g., fragments derived from the noncoding regions of SEQ ID NO:1, SEQ ID NO:3, SEQ ID NO:6, SEQ ID NO:8, or SEQ ID NO:10, having a length of at least 20 bases, preferably at least 30 bases.

The TRASH sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such TRASH probes can be used to identify tissue by species and/or by organ type.

In a similar fashion, these reagents, e.g., TRASH primers or probes can be used to screen tissue culture for contamination (i.e. screen for the presence of a mixture of different types of cells in a culture).

C. Predictive Medicine

The present invention also pertains to the field of predictive medicine in which diagnostic assays, prognostic assays, and monitoring clinical trails are used for prognostic (predictive) purposes to thereby treat an individual prophylactically. Accordingly, one aspect of the present invention relates to diagnostic assays for determining TRASH protein and/or nucleic acid expression as well as TRASH activity, in the context of a biological sample (e.g., blood, serum, cells, tissue) to thereby determine whether an individual is afflicted with a disease or disorder, or is at risk of developing a disorder, associated with aberrant TRASH expression or activity. The invention also provides for prognostic (or predictive) assays for determining whether an individual is at risk of developing a disorder associated with TRASH protein, nucleic acid expression or activity. For example, mutations in a TRASH gene can be assayed in a biological sample. Such assays can be used for prognostic or predictive purpose to thereby prophylactically treat an individual prior to the onset of a disorder characterized by or associated with TRASH protein, nucleic acid expression or activity.

Another aspect of the invention pertains to monitoring the influence of agents (e.g., drugs, compounds) on the expression or activity of TRASH in clinical trials.

These and other agents are described in further detail in the following sections.

1. Diagnostic Assays

An exemplary method for detecting the presence or absence of TRASH in a biological sample involves obtaining a biological sample from a test subject and contacting the biological sample with a compound or an agent capable of detecting TRASH protein or nucleic acid (e.g., mRNA, genomic DNA) that encodes TRASH protein such that the presence of TRASH is detected in the biological sample. A preferred agent for detecting TRASH mRNA or genomic DNA is a labeled nucleic acid probe capable of hybridizing to TRASH mRNA or genomic DNA. The nucleic acid probe can be, for example, a full-length TRASH nucleic acid, such as the nucleic acid of SEQ ID NO:1, or a portion thereof, such as an oligonucleotide of at least 15, 30, 50, 100, 250 or 500 nucleotides in length and sufficient to specifically hybridize under stringent conditions to TRASH mRNA or genomic DNA. Other suitable probes for use in the diagnostic assays of the invention are described herein.

A preferred agent for detecting TRASH protein is an antibody capable of binding to TRASH protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect TRASH mRNA, protein, or genomic DNA in a biological sample in vitro as well as in vivo. For example, in vitro techniques for detection of TRASH mRNA include Northern hybridizations and in situ hybridizations. In vitro techniques for detection of TRASH protein include enzyme linked immunosorbent assays (ELISAs), Western blots, immunoprecipitations and immunofluorescence. In vitro techniques for detection of TRASH genomic DNA include Southern hybridizations. Furthermore, in vivo techniques for detection of TRASH protein include introducing into a subject a labeled anti-TRASH antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting TRASH protein, mRNA, or genomic DNA, such that the presence of TRASH protein, mRNA or genomic DNA is detected in the biological sample, and comparing the presence of TRASH protein, mRNA or genomic DNA in the control sample with the presence of TRASH protein, mRNA or genomic DNA in the test sample.

The invention also encompasses kits for detecting the presence of TRASH in a biological sample. For example, the kit can comprise a labeled compound or agent capable of detecting TRASH protein or mRNA in a biological sample; means for determining the amount of TRASH in the sample; and means for comparing the amount of TRASH in the sample with a standard. The compound or agent can be packaged in a suitable container. The kit can further comprise instructions for using the kit to detect TRASH protein or nucleic acid.

2. Prognostic Assays

The diagnostic methods described herein can furthermore be utilized to identify subjects having or at risk of developing a disease or disorder associated with aberrant TRASH expression or activity. For example, the assays described herein, such as the preceding diagnostic assays or the following assays, can be utilized to identify a subject having or at risk of developing a disorder associated with TRASH protein, nucleic acid expression or activity such as an immune disorder, or a differentiative or developmental disorder. Alternatively, the prognostic assays can be utilized to identify a subject having or at risk for developing an inflammatory or immune disease. Thus, the present invention provides a method for identifying a disease or disorder associated with aberrant TRASH expression or activity in which a test sample is obtained from a subject and TRASH protein or nucleic acid (e.g, mRNA, genomic DNA) is detected, wherein the presence of TRASH protein or nucleic acid is diagnostic for a subject having or at risk of developing a disease or disorder associated with aberrant TRASH expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant TRASH expression or activity. For example, such methods can be used to determine whether a subject can be effectively treated with an agent for a disorder, such as an immune disorder, or a differentiative or developmental disorder. Alternatively, such methods can be used to determine whether a subject can be effectively treated with an agent for an immune system disease. Thus, the present invention provides methods for determining whether a subject can be effectively treated with an agent for a disorder associated with aberrant TRASH expression or activity in which a test sample is obtained and TRASH protein or nucleic acid is detected (e.g., wherein the presence of TRASH protein or nucleic acid is diagnostic for a subject that can be administered the agent to treat a disorder associated with aberrant TRASH expression or activity.)

The methods of the invention can also be used to detect genetic alterations in a TRASH gene, thereby determining if a subject with the altered gene is at risk for a disorder characterized by aberrant development, aberrant immune responsiveness, an aberrant inflammatory response or an aberrant hematopoietic response. In preferred embodiments, the methods include detecting, in a sample of cells from the subject, the presence or absence of a genetic alteration characterized by at least one of an alteration affecting the integrity of a gene encoding a TRASH-protein, or the mis-expression of the TRASH gene. For example, such genetic alterations can be detected by ascertaining the existence of at least one of 1) a deletion of one or more nucleotides from a TRASH gene; 2) an addition of one or more nucleotides to a TRASH gene; 3) a substitution of one or more nucleotides of a TRASH gene, 4) a chromosomal rearrangement of a TRASH gene; 5) an alteration in the level of a messenger RNA transcript of a TRASH gene, 6) aberrant modification of a TRASH gene, such as of the methylation pattern of the genomic DNA, 7) the presence of a non-wild type splicing pattern of a messenger RNA transcript of a TRASH gene, 8) a non-wild type level of a TRASH-protein, 9) allelic loss of a TRASH gene, and 10) inappropriate post-translational modification of a TRASH-protein. As described herein, there are a large number of assay techniques known in the art which can be used for detecting alterations in a TRASH gene. A preferred biological sample is serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the TRASH-gene (see Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a TRASH gene under conditions such that hybridization and amplification of the TRASH-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et al., 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a TRASH gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in TRASH can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in TRASH can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the TRASH gene and detect mutations by comparing the sequence of the sample TRASH with the corresponding wild-type (control) sequence. Examples of sequencing reactions include those based on techniques developed by Maxim and Gilbert ((1977) PNAS 74:560) or Sanger ((1977) PNAS 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the TRASH gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type TRASH sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl Acad Sci USA 85:4397; Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in TRASH cDNAs obtained from samples of cells. For example, the mut Y enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a TRASH sequence, e.g., a wild-type TRASH sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in TRASH genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat Res 285:125-144; and Hayashi (1992) Genet Anal Tech Appl 9:73-79). Single-stranded DNA fragments of sample and control TRASH nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a TRASH gene.

Furthermore, any cell type or tissue in which TRASH is expressed may be utilized in the prognostic assays described herein.

3. Monitoring of Effects During Clinical Trials

Monitoring the influence of agents (e.g., drugs, compounds) on the expression or activity of TRASH (e.g., activation of a TRASH-dependent signal transduction pathway; cytolysis of certain tumor cell lines; modulation of secretion of inflammatory mediators/cytokines; modulation of the development or differentiation of a TRASH-expressing cell; modulation of the development or differentiation of a non- TRASH-expressing cell; or modulation of host resistance to infectious agents) can be applied not only in basic drug screening, but also in clinical trials. For example, the effectiveness of an agent determined by a screening assay as described herein to increase TRASH gene expression, protein levels, or upregulate TRASH activity, can be monitored in clinical trails of subjects exhibiting decreased TRASH gene expression, protein levels, or downregulated TRASH activity. Alternatively, the effectiveness of an agent determined by a screening assay to decrease TRASH gene expression, protein levels, or downregulate TRASH activity, can be monitored in clinical trails of subjects exhibiting increased TRASH gene expression, protein levels, or upregulated TRASH activity. In such clinical trials, the expression or activity of TRASH and, preferably, other genes that have been implicated in, for example, an immune response disorder can be used as a “read out” or markers of the immune responsiveness of a particular cell.

For example, and not by way of limitation, genes, including TRASH, that are modulated in cells by treatment with an agent (e.g., compound, drug or small molecule) which modulates TRASH activity (e.g., identified in a screening assay as described herein) can be identified. Thus, to study the effect of agents on immune response disorders, or developmental disorders, for example, in a clinical trial, cells can be isolated and RNA prepared and analyzed for the levels of expression of TRASH and other genes implicated in the immune response disorders, or developmental disorders, respectively. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of TRASH or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of a TRASH protein, mRNA, or genomic DNA in the preadministration sample; (iii) obtaining one or more post-administration samples from the subject; (iv) detecting the level of expression or activity of the TRASH protein, mRNA, or genomic DNA in the post-administration samples; (v) comparing the level of expression or activity of the TRASH protein, mRNA, or genomic DNA in the pre-administration sample with the TRASH protein, mRNA, or genomic DNA in the post administration sample or samples; and (vi) altering the administration of the agent to the subject accordingly. For example, increased administration of the agent may be desirable to increase the expression or activity of TRASH to higher levels than detected, i.e., to increase the effectiveness of the agent. Alternatively, decreased administration of the agent may be desirable to decrease expression or activity of TRASH to lower levels than detected, i.e. to decrease the effectiveness of the agent. According to such an embodiment, TRASH expression or activity may be used as an indicator of the effectiveness of an agent, even in the absence of an observable phenotypic response.

C. Methods of Treatment

The present invention provides for both prophylactic and therapeutic methods of treating a subject at risk of (or susceptible to) a disorder or having a disorder associated with aberrant TRASH expression or activity. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype.”) Thus, another aspect of the invention provides methods for tailoring an individual's prophylactic or therapeutic treatment with either the TRASH molecules of the present invention or TRASH modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant TRASH expression or activity, by administering to the subject an agent which modulates TRASH expression or at least one TRASH activity. Subjects at risk for a disease which is caused or contributed to by aberrant TRASH expression or activity can be identified by, for example, any or a combination of diagnostic or prognostic assays as described herein. Administration of a prophylactic agent can occur prior to the manifestation of symptoms characteristic of the TRASH aberrancy, such that a disease or disorder is prevented or, alternatively, delayed in its progression. Depending on the type of TRASH aberrancy, for example, a TRASH agonist or TRASH antagonist agent can be used for treating the subject. The appropriate agent can be determined based on screening assays described herein. The prophylactic methods of the present invention are further discussed in the following subsections.

2. Therapeutic Methods

Another aspect of the invention pertains to methods of modulating TRASH expression or activity for therapeutic purposes. The modulatory method of the invention involves contacting a cell with an agent that modulates one or more of the activities of TRASH protein activity associated with the cell. An agent that modulates TRASH protein activity can be an agent as described herein, such as a nucleic acid or a protein, a naturally-occurring target molecule of a TRASH protein, a peptide, a TRASH peptidomimetic, or other small molecule. In one embodiment, the agent stimulates one or more TRASH protein activity. Examples of such stimulatory agents include active TRASH protein and a nucleic acid molecule encoding TRASH that has been introduced into the cell. In another embodiment, the agent inhibits one or more TRASH protein activity. Examples of such inhibitory agents include antisense TRASH nucleic acid molecules and anti-TRASH antibodies. These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant expression or activity of a TRASH protein or nucleic acid molecule. In one embodiment, the method involves administering an agent (e.g., an agent identified by a screening assay described herein), or combination of agents that modulates (e.g., upregulates or downregulates) TRASH expression or activity. In another embodiment, the method involves administering a TRASH protein or nucleic acid molecule as therapy to compensate for reduced or aberrant TRASH expression or activity.

Stimulation of TRASH activity is desirable in situations in which TRASH is abnormally downregulated and/or in which increased TRASH activity is likely to have a beneficial effect. One example of such a situation is where a subject has a disorder characterized by aberrant immune responsiveness. Another example of such a situation is where a subject has a disorder characterized by aberrant differentiation or development.

3. Pharmacogenomics

The TRASH molecules of the present invention, as well as agents, or modulators which have a stimulatory or inhibitory effect on TRASH activity (e.g., TRASH gene expression) as identified by a screening assay described herein can be administered to individuals to treat (prophylactically or therapeutically) disorders (e.g, immune response disorders or developmental disorders) associated with aberrant TRASH activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a TRASH molecule or TRASH modulator as well as tailoring the dosage and/or therapeutic regimen of treatment with a TRASH molecule or TRASH modulator.

Pharmacogenomics deals with clinically significant hereditary variations in the response to drugs due to altered drug disposition and abnormal action in affected persons. See e.g., Eichelbaum, M., Clin Exp Pharmacol Physiol, 1996, 23(10-11) :983-985 and Linder, M. W., Clin Chem, 1997, 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug target is known (e.g., a TRASH protein or TRASH receptor of the present invention), all common variants of that gene can be identified in the population and a particular drug response can be associated with one or more genes.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a TRASH molecule or TRASH modulator of the present invention) indicates whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a TRASH molecule or TRASH modulator, such as a modulator identified by one of the exemplary screening assays described herein.

This invention is further illustrated by the following examples which should not be construed as limiting. The contents of all references, patents and published patent applications cited throughout this application are incorporated herein by reference.

### EXAMPLES

### Example 1

### Isolation and Characterization of Human TRASH cDNAs

In this example, the isolation of the gene encoding human TRASH (also referred to as “TANGO 118”) is described.

A human TRASH cDNA was identified by searching against a copy of the GenBank nucleotide database using the BLASTN™ program (BLASTN 1.3MP: Altschul et al., J. Mol. Bio. 215:403, 1990). Numerous clones that consisted mostly of 3′ reads and some that were 5′ reads within the 3′ untranslated region were found by this search. The sequences were analyzed against a non-redundant protein database with the BLASTX™ program, which translates a nucleic acid sequence in all six frames and compares it against available protein databases (BLASTX 1.3MP:Altschul et al., supra). This protein database is a combination of the Swiss-Prot, PIR, and NCBI GenPept protein databases. One clone was obtained from the IMAGE consortium, and fully sequenced. The additional sequencing of this clone extended the original EST by 865 nucleotides further 5′. The cDNA for this clone is approximately 1344 nucleotides in length and has an open reading frame of 753 nucleotides that is predicted to encode a protein of 250 amino acids.

The original first pass sequence of the clone showed homology to human TNF-α using the BLASTX™ program. The nucleotide sequence and predicted amino acid sequence are shown in FIG. 1 (corresponding to SEQ ID NO:1 and SEQ ID NO:2, respectively). The human TRASH protein (corresponding to amino acids 1-250 of the predicted amino acid sequence, SEQ ID NO:2) shows 21.0% identity to the human TNF-α protein and 24.6% identity to the human Tweak protein (see FIG. 2). The percent identity was calculated using the alignment generated using MegAlign™ sequence alignment software. The initial pairwise alignment step was performed using a Wilbur-Lipmann algorithm with a K-tuple of 1, a GAP penalty of 3, a window of 5, and diagonals saved set to=4. The multiple alignment step was performed using the Clustal algorithm with a PAM 250 residue weight Table, a GAP penalty of 10, and a GAP length penalty of 10.

This human TRASH protein contains a TNF signature motif, a TNF-like N-terminal signal transmembrane anchor for a type II membrane protein (corresponding to amino acids 1-44 of the predicted amino acid sequence, SEQ ID NO:2), two cysteine residues which may be disulphide linked (corresponding to amino acids 196 and 211 of the predicted amino acid sequence, SEQ ID NO:2), and two putative N-lined glycosylation sites (corresponding to amino acids 124 and 237 of the predicted amino acid sequence, SEQ ID NO:2). This human TRASH protein also contains three potential initiating ATG codons that would result in polypeptides of 250aa (SEQ ID NO:2), 233aa (SEQ ID NO:9), or 205aa (SEQ ID NO:11) encoding methionines at positions 1, 18, and 46 of SEQ ID NO:2, respectively. The predicted molecular weight for the 250aa TRASH is approximately 27.4 kDa.

A BLASTN™ search of the EST database revealed the following ESTs having significant homology to clone Accession # AA481449:

| | | Base Pairs | % | |
|---|---|---|---|---|
| EST Database hits | Species | Covered | Identity | Coding? |
| Accession # AA405973 | human | 730-1224 | 97 | yes |
| Accession # AA293679 | human | 1318-884 | 100 | yes |
| Accession # AA394070 | human | 1318-891 | 100 | yes |
| Accession # AA443577 | human | 939-515 | 99 | yes |

### Example 2

### Distribution of TRASH mRNA In Human Tissues Northern Blot Analysis

The expression of TRASH was analyzed using Northern blot hybridization. For analysis of human TRASH, the 1.3 kb insert of AA4481449 was used as a probe. The probe DNA was radioactively labeled with ³²P-dCTP using the Prime-It kit™ (Stratagene, La Jolla, Cailf.) according to the instructions of the supplier. Filters containing mRNA (human MTNI and MTNII and murine embryo MTN from Clontech, Palo Alto, Calif.) were probed in ExpressHyb™ hybridization solution (Clontech) and washed at high stringency according to manufacturer's recommendations.

Expression was found predominantly in the peripheral blood leukocytes where a message of approximately 1.5 kb transcript was observed. In addition, a 1.5 kb transcript was observed in the spleen and lymph nodes and lung. A slightly larger message, approximately 1.7 kb was observed in the colon, spleen, and kidney.

### Example 3

### Expression of Recombinant TRASH Protein in Bacterial Cells

TRASH can be expressed as a recombinant glutathione-S-transferase (GST) fusion polypeptide in E. coli and the fusion polypeptide can be isolated and characterized. Specifically, TRASH is fused to GST and this fusion polypeptide is expressed in E. coli, e.g., strain PEB199. As the murine TRASH protein is predicted to be approximately 27.4 kDa and the GST is predicted to be approximately 26 kDa, the fusion polypeptide is predicted to be approximately 53.4 kDa in molecular weight. Expression of the GST-TRASH fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 4

### Expression of Recombinant TRASH Protein in COS Cells

To express the TRASH gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire TRASH protein and a HA tag (Wilson et al. (1984) Cell 37:767) fused in-frame to its 3′ end of the fragment is cloned into the polylinker region of the vector, thereby placing the expression of the recombinant protein under the control of the CMV promoter.

To construct the plasmid, the TRASH DNA sequence is amplified by PCR using two primers. The 5′ primer contains the restriction site of interest followed by approximately twenty nucleotides of the TRASH coding sequence starting from the initiation codon; the 3′ end sequence contains complementary sequences to the other restriction site of interest, a translation stop codon, the HA tag and the last 20 nucleotides of the TRASH coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the TRASH gene is inserted in the correct orientation. The ligation mixture is transformed into E. coli cells (strains HB101, DH5a, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the TRASH-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the TRASH protein is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated proteins are then analyzed by SDS-PAGE.

Alternatively, DNA containing the TRASH coding sequence is cloned directly into the polylinker of the pCDNA/Amp vector using the appropriate restriction sites. The resulting plasmid is transfected into COS cells in the manner described above, and the expression of the TRASH protein is detected by radiolabelling and immunoprecipitation using a TRASH specific monoclonal antibody

### Example 5

### Retroviral Delivery of TRASH

The entire open reading frame of TRASH can be subcloned into the retroviral vector MSCVneo, described in Hawley et al. (1994) Gene Therapy 1:136-138. Cells (293Ebna, Invitrogen) are then transiently transfected with the TRASH construct and with constructs containing viral regulatory elements, to produce high titre retrovirus containing the TRASH gene. The virus is then used to transfect mice. These mice are then tested for any gross pathology and for changes in their immune response using standard assays.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. An isolated nucleic acid molecule which encodes a TRASH protein, comprising a nucleotide sequence at least about 60% homologous to a nucleotide sequence of SEQ ID NO:3, or a complement thereof.

2. The isolated nucleic acid molecule of claim 1, further comprising nucleotides 1 272 of SEQ ID NO:1.

3. The isolated nucleic acid molecule of claim 1, further comprising nucleotides 1026-1344 of SEQ ID NO:1.

4. An isolated nucleic acid molecule which encodes a TRASH protein, comprising a nucleotide sequence at least about 60% homologous to a nucleotide sequence of SEQ ID NO:1, SEQ ID NO:6, SEQ ID NO:8, SEQ ID NO:10, or a complement thereof.

5. The isolated nucleic acid molecule of any of claims 1 or 4 which specifically detects a TRASH nucleic acid molecule relative to a nucleic acid molecule encoding a non-TRASH protein.

6. An isolated nucleic acid molecule comprising a nucleotide sequence encoding a protein which comprises an amino acid sequence at least about 60% homologous to the amino acid sequence of SEQ ID NO:2, SEQ ID NO:7, SEQ ID NO:9, or SEQ ID NO:11.

7. An isolated nucleic acid molecule encoding a TRASH protein, comprising a nucleotide sequence which hybridizes under stringent hybridization conditions to a nucleic acid molecule comprising the nucleotide sequence of SEQ ID NO:3, SEQ ID NO:6, SEQ ID NO:8, or SEQ ID NO:10.

8. An isolated nucleic acid molecule encoding a TRASH protein, comprising a nucleotide sequence which hybridizes under stringent hybridization conditions to a nucleic acid molecule comprising the nucleotide sequence of SEQ ID NO:1.

9. An isolated nucleic acid molecule comprising a nucleotide sequence which hybridizes under stringent hybridization conditions to a nucleic acid molecule comprising nucleotides 1-1025 of SEQ ID NO:1.

10. An isolated nucleic acid molecule comprising a nucleotide sequence which hybridizes under stringent hybridization conditions to a nucleic acid molecule comprising nucleotides 273-1344 of SEQ ID NO:1.

11. An isolated nucleic acid molecule at least 500 nucleotides in length which hybridizes under stringent hybridization conditions to a nucleic acid molecule comprising the nucleotide sequence of SEQ ID NO:1, SEQ ID NO:3, SEQ ID NO:6, SEQ ID NO:8, or SEQ ID NO:10.

12. An isolated nucleic acid molecule which is antisense to the nucleic acid molecule of any of claims 1, 4, 8, 9, or 10.

13. A vector comprising the nucleic acid molecule of any of claims 1, 4, 6, 7, or 8.

14. The vector of claim 13, which is a recombinant expression vector.

15. A host cell containing the vector of claim 14.

16. A method for producing TRASH protein comprising culturing the host cell of claim 15 in a suitable medium until TRASH protein is produced.

17. The method of claim 16, further comprising isolating TRASH protein from the medium or the host cell.

18. A nonhuman transgenic animal which contains cells carrying a transgene encoding TRASH protein.

19. A nonhuman homologous recombinant animal which contains cells having an altered TRASH gene.

20. An isolated TRASH protein comprising an amino acid sequence at least about 60% homologous to the amino acid sequence of SEQ ID NO:2, SEQ ID NO:7, SEQ ID NO:9, or SEQ ID NO:11.

21. An isolated TRASH protein which is encoded by a nucleic acid molecule comprising a nucleotide sequence at least about 60% homologous to a nucleotide sequence of SEQ ID NO:3, SEQ ID NO:6, SEQ ID NO:8, SEQ ID NO:10 or a complement thereof.

22. An isolated TRASH protein which is encoded by a nucleic acid molecule comprising a nucleotide sequence at least about 60% homologous to a nucleotide sequence of SEQ ID NO:1, SEQ ID NO:3, SEQ ID NO:6, SEQ ID NO:8, SEQ ID NO:10, or a complement thereof.

23. An isolated TRASH protein which is encoded by a nucleic acid molecule comprising a nucleotide sequence which hybridizes under stringent hybridization conditions to a nucleic acid molecule comprising the nucleotide sequence of SEQ ID NO:1, SEQ ID NO:3, SEQ ID NO:6, SEQ ID NO:8, or SEQ ID NO:10.

24. An isolated protein comprising an amino acid sequence sufficiently homologous to the amino acid sequence of SEQ ID NO:2, SEQ ID NO:7, SEQ ID NO:9, or SEQ ID NO:11, wherein the protein retains a TRASH biological activity.

25. The isolated protein of claim 24 comprising an amino acid sequence 60% homologous to an amino acid sequence of SEQ ID NO:2, SEQ ID NO:7, SEQ ID NO:9, or SEQ ID NO:11.

26. The isolated protein of any of claims 20-25, which is encoded by an amino acid molecule comprising an amino acid sequence at least about 55% homologous to the TNF signature motif of SEQ ID NO:2.

27. The isolated protein of any of claims 20-25, which is encoded by an amino acid molecule comprising an amino acid sequence at least about 50% homologous to the TNF-like N-terminal signal transmembrane anchor for a type II membrane protein of SEQ ID NO:2.

28. An isolated protein comprising the amino acid sequence of SEQ ID NO:2, SEQ ID NO:7, SEQ ID NO:9, or SEQ ID NO:11.

29. A fusion protein comprising a TRASH polypeptide operatively linked to a non-TRASH polypeptide.

30. The fusion protein of claim 29, wherein the TRASH polypeptide comprises a TNF signature motif.

31. The fusion protein of claim 29, wherein the non-TRASH polypeptide is an immunoglobulin domain.

32. An antibody that specifically binds TRASH.

33. The antibody of claim 32, which is monoclonal.

34. The antibody of claim 33, which is labeled with a detectable substance.

35. A pharmaceutical composition comprising the protein of any one of claims 20-25, or 29, and a pharmaceutically acceptable carrier.

36. A pharmaceutical composition comprising the antibody of claim 32 and a pharmaceutically acceptable carrier.

37. A method for modulating a cell-associated activity comprising contacting a cell with an agent which modulates TRASH protein activity or TRASH nucleic acid expression such that the cell-associated activity is altered relative to the cell-associated activity of the cell in the absence of the agent.

38. The method of claim 37, wherein the agent stimulates a TRASH protein activity or expression.

39. The method of claim 37, wherein the agent inhibits a TRASH protein activity or expression.

40. The method of claim 39, wherein the agent is an antisense TRASH nucleic acid molecule.

41. The method of claim 39, wherein the agent is an antibody that specifically binds to TRASH.

42. The method of claim 37, wherein the cell is present within a subject and the agent is administered to the subject.

43. A method for treating a subject having a disorder characterized by aberrant TRASH protein activity or nucleic acid expression comprising administering to the subject a TRASH modulator such that treatment of the subject occurs.

44. The method of claim 43, wherein the TRASH modulator is a small molecule.

45. The method of claim 43, wherein the TRASH modulator is a TRASH protein.

46. The method of claim 43 wherein the TRASH modulator is a nucleic acid molecule encoding a TRASH protein.

47. The method of claim 43, wherein the disorder is an immune response disorder.

48. The method of claim 43, wherein the disorder is an inflammatory disorder.

49. A method for detecting the presence of TRASH activity in a biological sample comprising contacting a biological sample with an agent capable of detecting an indicator of TRASH activity such that the presence of TRASH activity is detected in the biological sample.

50. The method of claim 49, wherein the agent detects TRASH mRNA.

51. The method of claim 49, wherein the agent is a labeled nucleic acid probe capable of hybridizing to TRASH mRNA.

52. The method of claim 49, wherein the agent detects TRASH protein.

53. The method of claim 49, wherein the agent is a labeled antibody capable of specifically binding to TRASH protein.

54. A kit for detecting the presence of TRASH activity in a biological sample comprising an agent capable of detecting an indicator of TRASH activity in a biological sample.

55. The kit of claim 54, wherein the agent is a nucleic acid probe capable of hybridizing to TRASH mRNA.

56. The kit of claim 54, wherein the agent is an antibody capable of specifically binding to TRASH protein.

57. The kit of claim 54, further comprising instructions for use.

58. A diagnostic assay for identifying a genetic alteration in a cell sample, the presence or absence of the genetic alteration characterized by at least one of (i) aberrant modification or mutation of a gene encoding a TRASH protein, and (ii) mis-regulation of said gene or (iii) aberrant post-translational modification of a TRASH protein.

59. The assay of claim 58, wherein detecting said alteration includes:
a. providing a reagent comprising a diagnostic probe of claim 10, 11, or 12;
b. combining said reagent with nucleic acid of said cell sample; and
c. detecting, by hybridization of said probe to said cellular nucleic acid, the existence of at least one of a deletion of one or more nucleotides from said gene, an addition of one or more nucleotides to said gene, a substitution of one or more nucleotides of said gene, a gross chromosomal rearrangement of all or a portion of said gene, a gross alteration in the level of an mRNA transcript of said gene, or a non-wild type splicing pattern of an mRNA transcript of said gene.

60. The assay of claim 58, wherein detecting said alteration includes:
a. providing a reagent comprising two diagnostic probes;
b. combining said reagent with nucleic acid of said cell sample; and
c. detecting, by amplification or lack of amplification of said cellular nucleic acid, the absence or existence of said alteration.

61. A method for identifying a compound that modulates the activity of a TRASH protein, comprising:
a. providing a indicator composition comprising a protein having TRASH activity;
b. contacting the indicator composition with a test compound; and
c. determining the effect of the test compound on TRASH activity in the indicator composition to thereby identify a compound that modulates the activity of a TRASH protein.


SEQUENCE LISTING (12 sequences)

SEQ ID NO: 1 - DNA (cDNA), 1344 bp; single-stranded, linear
  CDS 273..1022
gaattcggaacgaggggaacctaattctcctgaggctgagggagggtggagggtctcaaggcaacgctggccccacgacggagtgccaggagcactaacagtacccttagcttgctttcctcctccctcctttttattttcaagttcctttttatttctccttgcgtaacaaccttcttcccttctgcaccactgcccgtacccttacccgccccgccacctccttgctaccccactcttgaaaccacagctgttggcagggtccccagctcatgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgtgattgtgttataaaaagtggctcccagcttggaagaccagggtgggtacatactggagacagccaagagctgagtatataaaggagagggaatgtgcaggaacagaggcatcttcctgggtttggctccccgttcctcacttttcccttttcattcccaccccctagactttgattttacggatatcttgcttctgttccccatggagctccgaattcttgcgtgtgtgtagatgaggggcgggggacgggcgccaggcattgttcagacctggtcggggcccactggaagcatccagaacagcaccaccatctagcggcc

SEQ ID NO: 2 - protein, 250 aa [sequence omitted]

SEQ ID NO: 3 - DNA (cDNA), 754 bp; single-stranded, linear
atgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgtgat

SEQ ID NO: 4 - protein, 17 aa
  fragment type: internal
VRIQDAGVYLLYSQVLF

SEQ ID NO: 5 - protein, 49 aa
  fragment type: internal
MPASSPFLLAPKGPPGNMGGPVREPALSVALWLSWGAALGAVACAMALL

SEQ ID NO: 6 - DNA (cDNA), 603 bp; single-stranded, linear
  CDS 1..603
acccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactg

SEQ ID NO: 7 - protein, 201 aa
TQQTELQSLRREVSRLQGTGGPSQNGEGYPWQSLPEQSSDALEAWENGERSRKRRAVLTQKQKKQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 8 - DNA (cDNA), 699 bp; single-stranded, linear
  CDS 1..699
atggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactg

SEQ ID NO: 9 - protein, 233 aa [sequence omitted]

SEQ ID NO: 10 - DNA (cDNA), 615 bp; single-stranded, linear
  CDS 1..615
atggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactg

SEQ ID NO: 11 - protein, 205 aa [sequence omitted]

SEQ ID NO: 12 - protein, 14 aa
  Protein 2: /note= “Xaa is any amino acid.”
  Protein 4: /note= “Xaa is between 1 and 3 amino acids.”
  Protein 10: /note= “Xaa is between 1 and 2 amino acids.”
  Protein 13: /note= “Xaa is any amino acid.”
  fragment type: N-terminal
VXIXGVYLLXEVXF